(19) United States
(12) Patent Application Publication
(10) Pub. No.: US 2020/0110093 A1
(43) Pub. Date: Apr. 9, 2020

(54) METHOD FOR TREATING INTESTINAL GLUTAMINE SYNTHETASE ACTIVITY DEFICIENCY

(71) Applicant: New Biotic, Inc., Rowland Heights, CA (US)

(72) Inventors: Victoria Jung-Pan LIANG, Walnut, CA (US); Jocelyn ShinWei ANG, Rowland Heights, CA (US); Andrew Celino GACUYA, Chino Hills, CA (US); Sam Poon ANG, Rowland Heights, CA (US)

(21) Appl. No.: 16/700,189

(22) Filed: Dec. 2, 2019

Related U.S. Application Data
(63) Continuation of application No. 16/223,451, filed on Dec. 18, 2018.
Application No. 16/223,451 is a continuation of application No. PCT/US2018/062697, filed on Nov. 28, 2018.
(60) Provisional application No. 62/591,879, filed on Nov. 29, 2017.

(51) Int. Cl. G01N 33/68; A61K 35/741; B01L 3/00; A61P 25/00; G01N 33/94

(52) U.S. Cl. CPC G01N 33/68; A61K 35/741; G01N 2800/28; A61P 25/00; G01N 33/9406; B01L 3/5027

(57) ABSTRACT

The present invention relates to a method for monitoring intestinal glutamine synthetase levels in a mammal, particularly in a human subject, and is useful for detecting intestinal glutamine synthetase deficiency. The method is based on determining glutamate levels in the subject under controlled fasting and postprandial conditions after administration of a predetermined quantity of a glutamate containing protein composition. The method is useful for quantifying the ability of the mammal to metabolize dietary glutamate as a diagnostic marker for predicting the onset of or propensity for developing a central nervous system (CNS), psychotic, or neurological disorder, associated with glutamate toxicity. The method is also useful for designing regimens for rectifying glutamine synthetase deficiency levels in a mammal subject in order to treat or prevent such a disorder. This method and its corresponding quantification can be derived manually using data from current laboratory equipment, bio test chips, or it can be automated into a medical device or a laboratory apparatus complete with hardware and software for measurements with computational output showing quantification, diagnostic range or deficiency levels. Another advantage of this method is that because it detects glutamate toxicity, it can potentially detect and prevent the onset of neurological disease early on, before physical symptoms are manifested.

### FIELD OF THE INVENTION

The present invention relates to a method for monitoring intestinal glutamine synthetase (GS) levels in a mammal, particularly in a human subject, and is useful for detecting intestinal glutamine synthetase deficiency. The method is based on determining glutamate levels in the subject under controlled fasting and postprandial conditions after administration of a predetermined quantity of a glutamate containing protein composition. The method is useful for quantifying the ability of the mammal to metabolize dietary glutamate as a diagnostic marker for predicting the onset of or propensity for developing a central nervous system (CNS), psychotic, or neurological disorder, associated with glutamate toxicity. The method is also useful for designing regimens for rectifying glutamine synthetase deficiency levels in a mammal subject in order to treat or prevent such a disorder. This method and its corresponding quantification can be derived manually using data from current laboratory equipment, bio test chips, or it can be automated into a medical device or a laboratory apparatus complete with hardware and software for measurements with computational output showing quantification, diagnostic range or deficiency levels. Another advantage of this method is that because it detects glutamate toxicity, it can potentially detect and prevent the onset of neurological disease early on, before physical symptoms are manifested.

### BACKGROUND OF THE INVENTION

For many disease conditions such as cancer, tumors, liver, kidney, blood or genetic disorders, there are distinct biomarkers and blood tests to confirm a diagnosis. However, there are no precise biomarkers or a single reliable blood test available to properly diagnose a broad class of neurological and psychiatric conditions. In the case of a disease such as amyotrophic lateral sclerosis (ALS) or Parkinson's disease (PD), it often requires numerous medical examinations and tests to diagnose whether a patient has these conditions. The diagnosis process can include physical examinations, blood tests, and imaging procedures, such as magnetic resonance imagining (MRI). It is important to rule out other conditions and false diagnoses. The diagnostic process can typically take 9-12 months from the time symptoms are first observed. There are no blood tests that can positively diagnose these conditions, nor is there an efficient way to monitor the efficacy of a particular treatment. For rapidly advancing fatal diseases such as ALS, where the median survival time after diagnosis is about 31.8 months, a definitive biomarker or blood test pinpointing the disease could potentially open possibilities for early intervention, thereby saving lives.

Several studies have shown that patients with ALS (Andreaou, et al., 2008), Alzheimer's, (Miulli, Norwell, & Schwartz, 1993) Parkinson's (Iwasaki, Ikeda, Shojima, & Kinoshita, 1992), and multiple sclerosis (Westall, Hawkins, Ellison, & Myers, 1980) have increased glutamate (i.e. glutamic acid) levels in the plasma compared to healthy control patients, suggesting a systemic defect of glutamate metabolism as an underlying cause of the disease. Systemically defective metabolism of glutamate has long been suspected as a primary cause for ALS (Plaitakis & Caroscio, 1987). Other neurological diseases related to high levels of glutamate-induced toxicity include: autism (Shimmura, et al., 2011), schizophrenia (Ivanovaa, Boykoa, Yu., Krotenkoa, Semkea, & Bokhana, 2014), epilepsy (Rainesalo, Keranen, Palmio, Peltola, Oja, & Saransaari, 2004), Alzheimer's (Miulli, Norwell, & Schwartz, 1993), and psychotic diseases (Ivanovaa, Boykoa, Yu., Krotenkoa, Semkea, & Bokhana, 2014). Furthermore, using functional magnetic resonance imaging in a rat model of stroke, Campos and colleagues showed that decreasing plasma glutamate levels with blood glutamate scavengers was associated with a significant decrease of glutamate in the brain and correlated with neurological improvement. (Campos, et al., 2011). A similar study by Leibowitz and associates corroborated decreased blood glutamate concentration being associated with an improved neurological outcome (Leibowitz, Boyko, Shapira, & Zlotnik, 2012).

Glutamate is a major neurotransmitter of the human central nervous system and is among the most abundant amino acids in the body. The amino acid accounts for approximately 90 percent of the total neurotransmitter activity in the brain. The beneficial effects of glutamate are greatly dependent on strict homeostasis, by maintaining the concentration of glutamate in the brain's extracellular fluid (ECF) within the normal range of 0.3-2 μM/L) (Leibowitz, Boyko, Shapira, & Zlotnik, 2012). Animal models and human clinical studies reveal the association of pathologically elevated ECF glutamate levels and several acute and chronic neurodegenerative disorders, including stroke, traumatic brain injury (TBI), intracerebral hemorrhage, brain hypoxia, amyotrophic lateral sclerosis (ALS) (Andreaou, et al., 2008), dementia, and others. These disorders are characterized by a several hundred-fold elevation of glutamate concentration in the brain's ECF facilitated by a breakdown of the blood brain barrier (BBB), thus permitting free movement of glutamate between the blood plasma and brain extracellular fluid, along its concentration gradient. (Leibowitz, Boyko, Shapira, & Zlotnik, 2012).

Therefore, in our attempt to find an effective biomarker for neurological disease, we sought to indirectly measure the activity of GS in the gut. Our proposed biomarker measures serum glutamate level before and after intake of a predetermined amount of dietary glutamate. By measuring serum glutamate before and after ingestion of dietary glutamate, the efficiency of intestinal GS activity can be quantified since GS is the only enzyme that can perform this function.

We believe that intestinal GS activity is even more indicative of disease onset than serum GS level because it is elevated serum glutamate levels, the result of deficient GS activity in the intestines, which ultimately results in glutamate toxicity and its related neurological and psychotic diseases. This method is reliable, repeatable and effective because it allows us to bypass the biological complexity that has yet to be understood by simply calculating the activity of GS.

It is seen that elevated serum glutamate levels and the compromised ability to metabolize dietary glutamate to glutamine present potentially serious health issues. It is therefore apparent from the aforementioned literature that there is an urgent need to develop reliable diagnostic methods to quantify the effectiveness of glutamate metabolism from the dietary intake in a human subject in order to predict the onset or progression of many of the aforementioned neurological disease states. Furthermore, such diagnostic methods would provide a means to design effective treatment regimens and to monitor the efficacy of treatment. Another advantage of this method is that because it detects glutamate toxicity, it can potentially detect and prevent the onset of neurological disease early on, maybe even at 20 years of age.

### SUMMARY OF THE INVENTION

In the present invention, we have developed a method to quantify and monitor the efficiency of glutamate metabolism as a biomarker to measure glutamine synthetase deficiency to track the progression of, or predict the onset, or severity of various neurological conditions. These conditions include, but are not limited to, amyotrophic lateral sclerosis, Parkinson's disease, Alzheimer's disease, multiple sclerosis, dementia, peripheral neuropathy, restless legs syndrome, and a whole host of other psychiatric and related conditions associated with glutamate toxicity, such as anxiety disorders, autism, obsessive compulsive disorder (OCD), major depressive disorders, bipolar disorders, and schizophrenia. The present invention is achieved through the monitoring of glutamate levels of a human patient at two different time points to obtain both fasting and postprandial serum glutamate levels under a controlled regimen involving fasting followed by ingestion of a predetermined, standardized high glutamate-containing liquid meal or suspension. The methodology thereby provides a means for monitoring intestinal glutamine synthetase activity, particularly to identify a decreased activity which can indicate the risk of glutamate toxicity by a failure to properly metabolize glutamate to glutamine.

In another embodiment, the present invention relates to a method for monitoring intestinal glutamine synthetase activity in a human subject at two or more selected time points, comprising the steps of:

(a) fasting the patient, except for water, for a period of at least about 12 hours;

(b) withdrawing by venipuncture from the patient a first (fasting) blood sample;

(c) transferring the first blood sample to a first container, optionally containing an anticoagulant pre-cooled between about 0° C. to about 5° C.;

(d) orally administering to the patient an aqueous solution or suspension comprising the equivalent of about 5 to about 15 grams of glutamic acid (glutamate);

(e) about 15 minutes to about 90 minutes after the administration of the aqueous solution or suspension of step (d), withdrawing by venipuncture from the patient a second (post prandial) blood sample;

(f) transferring the second blood sample to a second container, optionally containing an anticoagulant pre-cooled between about 0° C. to about 5° C.;

(g) centrifuging each of the first and second blood samples to separate the blood serum from the blood platelets in the blood samples, to provide a first (fasting) serum sample and a second (post prandial) serum sample,

(h) deproteinization of each of the first serum sample and the second serum sample by the addition of a deproteinizing agent to each of the serum samples;

(i) centrifuging each of the serum samples from step (h) to separate the protein from the serum in the samples, to provide a first (fasting) protein free serum sample and a second (post prandial) protein free serum sample;

(j) analyzing the first and second protein free serum samples to determine the serum glutamate level of each sample; and

(k) comparing the serum glutamate levels from step (j) to indirectly determine the intestinal glutamine synthetase activity of the patient.

The present invention also relates to a method for monitoring intestinal glutamine synthetase activity in a human subject, comprising the steps of:

(i) providing a first (fasting) blood sample which is obtained from the subject at a first time point in a fasting state, wherein the subject is preferably fasted, except for water, for a period of at least about 12 hours;

(ii) providing a second (post prandial) blood sample which is obtained from the subject at a second time point that is about 15 minutes to about 90 minutes after oral administration of an aqueous solution or suspension comprising the equivalent of about 5 to about 15 grams of glutamic acid (glutamate) to the subject in the fasting state of step (i);

(iii) transferring the first blood sample to a first container, optionally containing an anticoagulant pre-cooled between about 0° C. to about 5° C.;

(iv) transferring the second blood sample to a second container, optionally containing an anticoagulant pre-cooled between about 0° C. to about 5° C.;

(v) centrifuging each of the first and second blood samples to separate the blood serum from the blood platelets in the blood samples, to provide a first (fasting) serum sample and a second (post prandial) serum sample,

(vi) deproteinization of each of the first serum sample and the second serum sample by the addition of a deproteinizing agent to each of the serum samples;

(vii) centrifuging each of the serum samples from step (vi) to separate the protein from the serum in the samples, to provide a first (fasting) protein free serum sample and a second (post prandial) protein free serum sample;

(viii) analyzing the first and second protein free serum samples to determine the serum glutamate level of each sample; and

(ix) comparing the serum glutamate levels from step (viii) to indirectly determine the intestinal glutamine synthetase activity of the patient.

In another embodiment, the present invention relates to a method wherein in step (k) or (ix) the intestinal glutamine synthetase activity of the patient is determined from the difference between the serum glutamate levels of each sample.

In another embodiment, the present invention relates to a method wherein in step (k) or (ix) the intestinal glutamine synthetase activity of the patient is determined from the ratio of the serum glutamate levels of each sample.

In another embodiment, the present invention relates to a method wherein in step (k) or (ix) the intestinal glutamine synthetase activity for the patient is determined as a ratio of intestinal glutamine synthetase deficiency by (A) determining the difference between the serum glutamate level in the second sample and the serum glutamate level in the first sample, (B) subtracting 30 μmol/liter from the result of step (A), and (C) dividing the result of step (B) by the approximate maximum serum glutamate level for a sample population. It should be noted that the maximum serum glutamate level for a sample population can vary. Values of over 100 μmol/liter and over 150 μmol/liter are possible. Such a value can be 157 μmol/liter.

In another embodiment, the present invention relates to a method comprising the further step (D) of step (k) multiplying the result of step (C) of step (k) by 100 to obtain a percentage of intestinal glutamine synthetase deficiency.

In another embodiment, the present invention relates to a method wherein in step (d) or (ii) the aqueous solution or suspension comprises the equivalent of about 70 mg/kg to about 225 mg/kg based on the weight of the patient of glutamic acid (glutamate).

In another embodiment, the present invention relates to a method wherein in step (d) or (ii) the aqueous solution or suspension comprises the equivalent of about 10 grams of glutamic acid (glutamate).

In another embodiment, the present invention relates to a method wherein in step (d) or (ii) the aqueous solution or suspension comprises the equivalent of about 150 mg/kg based on the weight of the patient of glutamic acid (glutamate).

In another embodiment, the present invention relates to a method wherein in step (d) or (ii) the aqueous suspension or solution is of a digestible protein.

In another embodiment, the present invention relates to a method wherein in step (d) or (ii) the aqueous suspension or solution of the digestible protein substantially free of glutamine.

In another embodiment, the present invention relates to a method wherein in step (d) or (ii) the aqueous suspension or solution is a solution or suspension of whey protein.

In another embodiment, the present invention relates to a method wherein in step (d) or (ii) the aqueous suspension or solution of the whey protein is substantially free of glutamine.

In another embodiment, the present invention relates to a method wherein in step (d) or (ii) the aqueous suspension or solution comprises about 75 grams [preferably about 50] of the whey protein suspended or dissolved in about 200 to about 250 ml of water or fruit juice.

In another embodiment, the present invention relates to a method wherein in step (d) or (ii) the fruit juice is apple juice.

In another embodiment, the present invention relates to a method wherein the time in step (e) or (ii) is about 60 minutes.

In another embodiment, the present invention relates to a method wherein in step (b) or (i) the first (fasting) blood sample has a volume of about 1 to about 10 ml and wherein in step (e) or (ii) the second (post prandial) blood sample has a volume of about 1 to about 10 ml.

In another embodiment, the present invention relates to a method wherein in step (b) or (i) the first (fasting) blood sample has a volume of about 5 ml and wherein in step (e) or (ii) the second (post prandial) blood sample has a volume of about 5 ml.

In another embodiment, the present invention relates to a method wherein the anticoagulant in step (c) or (iii) and the anticoagulant in step (f) or (iv) is selected from EDTA (ethylene diamine tetraacetic acid), lithium heparin, sodium citrate, and sodium heparin.

In another embodiment, the present invention relates to a method wherein the anticoagulant in step (c) or (iii) and the anticoagulant in step (f) or (iv) is EDTA (ethylene diamine tetraacetic acid).

In another embodiment, the present invention relates to a method wherein in step (g) or (v) the centrifuging is performed at about 17,000×g for about 10 minutes at about 0° C. to about 5° C. on each of the first blood sample and the second blood sample.

In another embodiment, the present invention relates to a method wherein in step (h) or (vi) the deproteinizing agent is selected from perchloric acid, trichloroacetic acid, and tungstic acid.

In another embodiment, the present invention relates to a method wherein in step (h) or (vi) the deproteinizing agent is perchloric acid.

In another embodiment, the present invention relates to a method wherein in step (h) or (vi) the deproteinizing agent is perchloric acid having a concentration of about 0.2 N to about 0.4 N and a volume of about 5 ml.

In another embodiment, the present invention relates to a method wherein in step (i) or (vii) the centrifuging is performed at about 19,000×g for about 10 minutes at about 0° C. to about 5° C. on each of the first blood sample and the second blood sample.

In another embodiment, the present invention relates to a method wherein the analysis in step (j) or (viii) is performed by an enzyme-linked immunosorbent assay (ELISA).

In another embodiment, the present invention relates to a method comprising the further step (I) of treating the human subject for intestinal glutamine synthetase activity deficiency or an abnormal elevated (excess) serum glutamate or a disease associated therewith or preventing progression of such disease if the difference between intestinal glutamine synthetase activity of the second sample and the intestinal glutamine synthetase activity of the first sample is greater than a predetermined value.

The present invention also relates to a method comprising diagnosing the subject with intestinal glutamine synthetase activity deficiency or an abnormal elevated (excess) serum glutamate or having or at risk for a disease associated therewith or its progression if the difference between intestinal glutamine synthetase activity of the second sample and the intestinal glutamine synthetase activity of the first sample is greater than a predetermined value.

In another embodiment, the present invention relates to a method comprising the further step (I) of treating the human subject for intestinal glutamine synthetase activity deficiency or an abnormal elevated (excess) serum glutamate or a disease associated therewith or preventing progression of such disease if the difference between the serum glutamate level in the second sample to the serum glutamate level in the first sample is greater than a predetermined value.

In another embodiment, the present invention relates to a method wherein the predetermined value is 60 μmol/liter of serum glutamate.

In another embodiment, the present invention relates to a method wherein the predetermined value is 30 μmol/liter of serum glutamate.

The present invention also relates to a method comprising diagnosing the subject with intestinal glutamine synthetase activity deficiency or an abnormal elevated (excess) serum glutamate or having or at risk for a disease associated therewith or its progression if the difference between intestinal glutamine synthetase activity of the second sample and the intestinal glutamine synthetase activity of the first sample is greater than a predetermined value.

In another embodiment, the present invention relates to a method comprising the further step (I) of treating the human subject for intestinal glutamine synthetase activity deficiency or an abnormal elevated (excess) serum glutamate or a disease associated therewith or preventing progression of such disease if the percent intestinal glutamine synthetase deficiency is greater than a predetermined value.

In another embodiment, the present invention relates to a method wherein the predetermined value is 19.11 percent.

In another embodiment, the present invention relates to a method wherein in step (I) the method of treating intestinal glutamine synthetase activity deficiency or an abnormal (excess) serum glutamate or a disease associated therewith or preventing progression of such disease is by increasing the intestinal glutamine synthetase activity in the patient.

The present invention also relates to use of an agent capable of increasing an intestinal glutamine synthetase activity for manufacturing a medicament for treating intestinal glutamine synthetase activity deficiency or an abnormal elevated (excess) serum glutamate or a disease associated therewith or preventing progression of such disease in a subject in need. In some embodiments, the agent is a probiotic to adjust the population of non-pathogenic glutamine synthetase producing bacteria in the small intestines of the subject. In some embodiments, the agent is a probiotic with a prebiotic to adjust the population of non-pathogenic glutamine synthetase producing bacteria in the small intestines of the subject. In some embodiment, the agent is for oral administration

In another embodiment, the present invention relates to a method wherein the treating method in step (I) comprises administering glutamine synthetase to the patient.

The present invention also relates to use of a glutamine synthetase for manufacturing a medicament for treating intestinal glutamine synthetase activity deficiency or a disease associated therewith or preventing progression of such disease in a subject in need.

In another embodiment, the present invention relates to a method wherein the method in step (I) comprises orally administering administering a probiotic to adjust the population of non-pathogenic glutamine synthetase producing bacteria in the small intestines of the patient.

In another embodiment, the present invention relates to a method wherein the method in step (I) comprises orally administering a probiotic with a prebiotic to adjust the population of non-pathogenic glutamine synthetase producing bacteria in the small intestines of the patient.

In another embodiment, the present invention relates to a method for treating a central nervous system or psychotic disorder.

In another embodiment, the present invention relates to a method wherein the neurological or psychotic disorder is selected from Alzheimer's disease, amyotrophic lateral sclerosis, autism, cerebral atrophy, dementia, epilepsy, major depressive disorders, multiple sclerosis, obsessive compulsive disorder, Parkinson's disease, peripheral neuropathy, restless legs syndrome, schizophrenia, stiff man syndrome, and stroke.

In another embodiment, the present invention relates to a method using hardware, a biochip, micro and nano-array technologies or equivalent, or in combination with chemical or radio isotope labeling techniques for automated measurement of serum glutamate levels, and complete with hardware and software for measurements with computational output showing quantification, diagnostic range of intestinal glutamine synthetase deficiency levels.

In another embodiment, the present invention relates to a medical device or apparatus for diagnosing glutamate levels in blood serum comprising the use of hardware, a biochip, micro and nano-array technologies or equivalent or in combination with chemical or radio isotopes and complete with hardware and software for measurements with computational output showing quantification, diagnostic range of intestinal glutamine synthetase deficiency levels.

In another aspect, the present invention relates to a kit for performing the method as described herein comprising an agent that is capable of specifically detecting glutamate in the samples, and instructions for performing the method.

In another aspect, the present invention relates to use of a biomarker for manufacturing a kit, wherein the biomarker is glutamate in a blood sample from a subject, said kit useful for quantifying intestinal glutamine synthetase activity, comprising obtaining a first (fasting) blood sample from the subject at a first time point in a fasting state; obtaining a second (post prandial) blood sample from the subject at a second time point that is about 15 minutes to about 90 minutes after oral administration of an aqueous solution or suspension comprising the equivalent of about 5 to about 15 grams of glutamic acid (glutamate) to the subject in the fasting state; analyzing the samples to obtain fasting and postprandial serum glutamate levels; and comparing the levels to determine the intestinal glutamine synthetase activity.

In another aspect, the present invention relates to use of a biomarker for manufacturing a kit, wherein

the intestinal glutamine synthetase activity of the subject is determined from the difference between the serum glutamate levels of each sample;

the intestinal glutamine synthetase activity of the subject is determined from the ratio of the serum glutamate levels of each sample; or

the intestinal glutamine synthetase activity for the subject is determined as a ratio of intestinal glutamine synthetase deficiency by (A) determining the difference between the serum glutamate level in the second sample and the serum glutamate level in the first sample, (B) subtracting 30 μmol/liter from the result of step (A), and (C) dividing the result of step (B) by the approximate maximum serum glutamate level (defined as the difference of the post prandial serum glutamate level minus the fasting serum glutamate level) for a sample population, and optionally (D) multiplying the result of step (C) by 100 to obtain a percentage of intestinal glutamine synthetase deficiency.

In another aspect, the present invention relates to use of a biomarker for manufacturing a kit, wherein

the difference between the serum glutamate level in the second sample to the serum glutamate level in the first sample greater than 30 μmol/liter of serum glutamate is indicative of intestinal glutamine synthetase activity deficiency or an abnormal elevated (excess) serum glutamate or having or at risk for a disease associated therewith or its progression, or

the percent intestinal glutamine synthetase deficiency greater than 19.11 percent is indicative of intestinal glutamine synthetase activity deficiency or an abnormal elevated (excess) serum glutamate or having or at risk for a disease associated therewith or its progression.

In another aspect, the present invention relates to a pharmaceutical composition for use in treating intestinal glutamine synthetase activity deficiency or a disease associated therewith or preventing progression of such disease in a subject in need, comprising an agent capable of increasing an intestinal glutamine synthetase activity in the subject and a pharmaceutically acceptable carrier.

In another aspect, the present invention relates to a pharmaceutical composition, wherein the agent is a probiotic to adjust the population of non-pathogenic glutamine synthetase producing bacteria in the small intestines of the subject.

In another aspect, the present invention relates to a pharmaceutical composition, wherein the agent is a probiotic with a prebiotic to adjust the population of non-pathogenic glutamine synthetase producing bacteria in the small intestines of the subject.

In another aspect, the present invention relates to a pharmaceutical composition for use in treating intestinal glutamine synthetase activity deficiency or a disease associated therewith or preventing progression of such disease in a subject in need, comprising a glutamine synthetase and a pharmaceutically acceptable carrier.

### Definitions

As used herein, the following terms have the indicated meanings unless expressly stated to the contrary.

The term “subject” means a human subject or patient or animal in need of diagnosis or treatment or intervention or prognosis a disease or condition e.g. for pain or pruritus, particularly neuropathic pain or pruritus.

The term “therapeutically effective” means an amount of the therapeutic agent needed to provide a meaningful or demonstrable benefit, as understood by medical practitioners, to a subject, such as a human patient or animal, in need of treatment.

The terms “treat,” “treating” or “treatment,” as used herein, include alleviating, abating or ameliorating the condition, e.g. the elevated serum glutamate level or the associated central nervous system condition, or preventing or reducing the risk of contracting the condition or exhibiting the symptoms of the condition, ameliorating or preventing the underlying causes of the symptoms, inhibiting the condition, arresting the development of the condition, relieving the condition, causing regression of the condition, or stopping the symptoms of the condition, either prophylactically and/or therapeutically.

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of ±5% around the cited value.

According to the present invention, glutamate, particular a glutamate level in a blood sample can be used as a marker for quantifying/measuring intestinal glutamine synthetase activity and/or for diagnosing intestinal glutamine synthetase activity deficiency or an abnormal elevated serum glutamate or occurrence or risk for a disease associated therewith or its progression. As used herein, a biological marker (or called biomarker or marker) is a characteristic that is objectively measured and evaluated as an indicator of normal or abnormal biologic processes/conditions, diseases, pathogenic processes, or responses to treatment or therapeutic interventions. Markers can include presence or absence of characteristics or patterns or collections of the characteristics which are indicative of particular biological processes/conditions. A marker is normally used for diagnostic and/or prognostic purposes. However, it may be used for therapeutic, monitoring, drug screening and other purposes described herein, including evaluation the effectiveness of a cancer therapeutic.

“Diagnosis” as used herein generally includes determination as to whether a subject is likely affected by a given disease, disorder or dysfunction. The skilled artisan often makes a diagnosis on the basis of one or more diagnostic indicators, i.e., a marker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

As used herein, an “abnormal elevated” level can refer to a level that is increased compared with a reference or control level. For example, an abnormal elevated level can be higher than a reference or control level by more than 10%, 20%, 30%, 40%, 50%, 60%, 70%, 80%, 90% or 100%, or 2-fold, 3-fold, 4-fold, 5-fold, 6-fold, 7-fold, 8-fold, 9-fold, 10-fold, 11-fold or more. A reference or control level can refer to the level measured in normal individuals that are not diseased.

As used herein, a material that is described as being “substantially free” of a substance includes less than 5% (w/w), less than 4%, less than 3% (w/w), less than 2% (w/w), less than 1% (w/w) or a non-detectable amount of the substance.

### DETAILED DESCRIPTION OF INVENTION

In the human body and most mammals, glutamate is metabolized to glutamine. The enzyme glutamine synthetase (GS) catalyzes the condensation of glutamate and ammonia to form glutamine as depicted by the following reaction:

Glutamate (Glu)+ATP+NH3→Glutamine (Gln)+ADP+Phosphate

In the event there is insufficient GS enzyme in the small intestines, only a portion of Glu will be converted into Gln. See Table 1.

TABLE 1

| | Glutamine | | |
|---|---|---|---|
| | Synthetase (GS) | Serum | Serum |
| Dietary | Enzyme Activity | Glutamine | Glutamate |
| Glutamate | in the Intestine | (Gln) | (Glu) |
| 10-15 g per meal | 100% GS activity | Increase in | No increase in |
| | | serum Gln | serum Glu |
| 10-15 g per meal | 0% GS activity | No increase in | Increase in serum |
| | | serum Gln | Glu |
| 10-15 g per meal | Partial GS | Partial increase | Partial increase in |
| | activity | in serum Gln | serum Glu |
| | | (e.g., X %) | (e.g., 1-X %) |

Based on this, we measure the serum glutamate levels after a 12 hour fast and subtract that from the postprandial serum glutamate level (1 to 1.5 hours after ingestion of a glutamate liquid diet to determine the efficiency of the GS enzyme in converting glutamate to glutamine). This result is then divided by the difference of postprandial and fasting serum glutamate baseline observed from a population of healthy subjects. We apply a factor of 30 μM/L for residual or baseline serum glutamate levels typically observed in healthy subjects into the expression below.

$$\frac{( {Glu}_{pp} - {Glu}_{f} ) - 30}{Max ( {Glu}_{pp} - {Glu}_{f} ) - 30} \times 100 \% = \% GS deficiency$$

The equation presented immediately above is for determining the percentage of glutamine synthetase deficiency where Glu refers to serum glutamate, f refers to fasting conditions, pp refers to postprandial conditions, and GS refers to glutamine synthetase enzyme.

The above equation uses the subject's measured serum glutamate levels to quantify deficiency in conversion of glutamate to glutamine and consequently, deficiency in glutamine synthetase. The level of serum glutamate when fasting is subtracted from the subject's postprandial serum glutamate level after ingesting a standardized amount of pure glutamate under clinical test conditions. Then 30 μM/L is subtracted to cancel out the increase in glutamate expected from the consumption of the pure glutamate. The difference in these values is then compared to the most severe case observed in the data pool. This is done by dividing the difference between values by the difference between the postprandial and fasting glutamate levels of the subject with the highest value for this measurement within the subject pool, which in this case is 187 μM/L. Afterwards, 30 μM/L is again subtracted to cancel out the expected increase in glutamate. In a healthy subject, the metabolism of glutamate to glutamine by glutamine synthetase should result in a value less than or equal to 30 μM/L, with standard error. Therefore, it was an added conditional in the above model that the calculated percentage of glutamine synthetase deficiency should be forced to 0% to mean no deficiency.

Our clinical observations of subjects with ALS showed that in our model, the calculated percentage of glutamine synthetase deficiency was a positive percentage, i.e. deficiency. Since the subject's score is compared to the subject with the highest difference between postprandial and fasting glutamate levels, precise numbers of glutamate synthetase deficiency ranging from 0 to 100% can be calculated. This shows that the ingested glutamate of the testing process is not being converted to glutamine, indicating that there is a deficiency of glutamine synthetase to do this conversion.

A score of 0% through this model implies that the subject is healthy and does not suffer from a glutamate synthetase deficiency. If the difference between their postprandial and fasting glutamate levels is less than or equal to 30 μM/L, then it implies that their bodies could successfully and efficiently metabolize the consumed glutamate into glutamine within the time between the collection of the two samples.

A score of 100% can only be achieved by the subject whose difference between their two glutamate levels was the highest recorded, and thus was used directly in the formula. This value will be updated to reflect an updated and expanded data pool if it is necessitated by a new subject with a higher calculated difference than the current subject.

Nakagawa and associates found that in healthy subjects the difference between the fasting serum glutamate and postprandial serum glutamate levels after consuming 14.5 g of dietary glutamate for an average 70 kg person, are within the levels from 33±16 μmol/L to 63±34 μmol/L (Nakagawa, Takahashi, & Suzuki, 1960). This serum glutamate differential in healthy subjects is consistent with previous findings, showing an approximately 2-fold increase in peak plasma glutamate levels compared to fasting levels after administration of a high protein meal containing 207 mg/kg of total glutamate (See, Stegink, L. D. et al., Factors Affecting Plasma Glutamate Levels in Normal Adults Subjects, pages 333-351, page 345, in Glutamic Acid: Advances in Biochemistry and Physiology, edited by L. J. Filer, Jr., et al. Raven Press, NY 1979.). In contrast, the differential observed in human subjects exhibiting a neurological condition associated with glutamate toxicity, is abnormally large, e.g., 271 to 340.4 μmol/L (see the Examples section). We have also shown that with patients exhibiting improvement of symptoms after treatment, both the fasting serum glutamate and postprandial serum glutamate levels decline, commensurate with a lessening of the severity of or with a regression of the neurological condition.

Furthermore, it is previously been found that there is a correlation of plasma glutamate levels with glutamate ingested in a meal system. See, Stegink, L. D. et al., 1979. For example, various high protein foods such as custard, hamburgers, and milk shakes contain relatively high levels of glutamate. The following Table A provides data from Stegink et al. on the intake of protein in g/kg and glutamate in mg/kg (for an average adult) and the observed plasma glutamate levels (μm/dl) for fasting, peak, and the range. As seen in the entries if the values of Peak−Fasting are subtracted, i.e. 6.3 minus 3.3 or 7.1 minus 4.1, this leaves a value of 3.0 μm/dl, which when multiplied by 10 gives a value of 30 μm/l.

TABLE A

| | Protein | | Total | | | |
|---|---|---|---|---|---|---|
| | intake | MSG added | glutamate | Plasma glutamate levels (μm/dl) | | |
| Meal | (g/kg) | (mg/kg) | (mg/kg) | Fasting^{a} | Peak^{a} | Range^{b} |
| Custard | 1.0 | 0 | 207 | 3.3 ± 1.6 | 6.3 ± 3.4 | 3-12 |
| (adults) | | | | | | |
| Hamburger - | 1.0 | 0 | 171 | 4.1 ± 1.8 | 7.1 ± 3.9 | 4-15 |
| milk shake | | | | | | |

N = 6,
^{a}Mean ± SD,
^{b}Peak values

For many disease conditions such as cancer, tumors, liver, kidney, blood or genetic disorders, there are distinct biomarkers and blood tests to confirm a diagnosis. However, there are no precise biomarkers nor a single reliable blood test available to properly diagnose a broad class of neurological and psychiatric conditions. In the case of a disease such as amyotrophic lateral sclerosis (ALS) or Parkinson's disease, it often requires numerous medical examinations and tests to diagnose whether a patient has these conditions. The diagnosis process can include physical examinations, blood tests, and imaging procedures, such as magnetic resonance imagining (MRI). It is important to rule out other conditions and false diagnoses. The diagnostic process can typically take 9-12 months from the time symptoms are first observed. There are no blood tests that can positively diagnose these conditions, nor is there an efficient way to monitor the efficacy of a particular treatment. For rapidly advancing fatal diseases such as ALS, where the median survival time after diagnosis is about 31.8 months, a definitive biomarker or blood test pinpointing the disease could potentially open possibilities for early intervention, thereby saving lives.

Several studies have shown that patients with ALS (Andreaou, et al., 2008), Alzheimer's, (Miulli, Norwell, & Schwartz, 1993) Parkinson's (Iwasaki, Ikeda, Shojima, & Kinoshita, 1992), and multiple sclerosis (Westall, Hawkins, Ellison, & Myers, 1980) have increased glutamate levels in the plasma compared to healthy control patients, suggesting a systemic defect of glutamate metabolism as an underlying cause of the disease. Systemically defective metabolism of glutamate has long been suspected as a primary cause for ALS (Plaitakis & Caroscio, 1987). Other neurological diseases related to high levels of glutamate-induced toxicity include: autism (Shimmura, et al., 2011), schizophrenia (Ivanovaa, Boykoa, Yu., Krotenkoa, Semkea, & Bokhana, 2014), epilepsy (Rainesalo, Keranen, Palmio, Peltola, Oja, & Saransaari, 2004), Alzheimer's (Miulli, Norwell, & Schwartz, 1993), and psychotic diseases (Ivanovaa, Boykoa, Yu., Krotenkoa, Semkea, & Bokhana, 2014). Furthermore, using functional magnetic resonance imaging in a rat model of stroke, Campos and colleagues showed that decreasing plasma glutamate levels with blood glutamate scavengers was associated with a significant decrease of glutamate in the brain and correlated with neurological improvement. (Campos, et al., 2011). A similar study by Leibowitz and associates corroborated decreased blood glutamate concentration being associated with an improved neurological outcome (Leibowitz, Boyko, Shapira, & Zlotnik, 2012).

Glutamate is a major neurotransmitter of the human central nervous system and is among the most abundant amino acids in the body. The amino acid accounts for approximately 90 percent of the total neurotransmitter activity in the brain. The beneficial effects of glutamate are greatly dependent on strict homeostasis, by maintaining the concentration of glutamate in the brain's extracellular fluid (ECF) within the normal range of 0.3-2 μM/L) (Leibowitz, Boyko, Shapira, & Zlotnik, 2012). Animal models and human clinical studies reveal the association of pathologically elevated ECF glutamate levels and several acute and chronic neurodegenerative disorders, including stroke, traumatic brain injury (TBI), intracerebral hemorrhage, brain hypoxia, amyotrophic lateral sclerosis (ALS) (Andreaou, et al., 2008), dementia and others. These disorders are characterized by a several hundred-fold elevation of glutamate concentration in the brain's ECF facilitated by a breakdown of the blood brain barrier (BBB), thus permitting free movement of glutamate between the blood plasma and brain extracellular fluid, along its concentration gradient. (Leibowitz, Boyko, Shapira, & Zlotnik, 2012).

The BBB is formed by an interacting network of endothelial cells, pericytes and astrocytes. Endothelial cells form the inner layer of blood vessels and are bound to each other by tight junctions, while pericytes enwrap the endothelial cells and help maintain homeostasis and hemostasis in the BBB. Lastly, astrocytes endfeet cover the pericytes and maintain the sanctity of the tight junctions through the secretion of growth factors. In addition to maintaining tight junctions, these growth factors also promote enzymatic systems and the polarization of transporters, including glutamate transporters. Astrocytes along the BBB also regulate the BBB's ionic concentration and astrocytic polarization through various protein and ion transporters in their endfeet, such as glucose receptors and K+ channels (Cabezas, et al., 2014). Once thought to be present in neurons but not astrocytes, astrocyte endfeet have been proven to contain both N-methyl-D-aspartate (NMDA) receptors and α-amino-3-hydroxy-5-methyl-4-isoxazolepropionic acid (AMPA) receptors (Dzamba, Honsa, & Anderova, 2013).

Both AMPA receptors (AMPAR) and NMDA receptors (NMDAR) are ionotropic transmembrane receptors that have agonist binding sites for glutamate. Once glutamate binds to an AMPAR, the receptor is activated and opens an ion channel permeable to sodium and potassium. In a neuron, if there is enough glutamate to activate the receptor for long enough, the influx of sodium and potassium will be great enough to depolarize the interior of the neuron, thus causing the magnesium ion blocking the NMDAR's ion channel to dislodge and allowing Ca²⁺ to flow through the glutamate-mediated ion channel. However, NMDARs in astrocytes are either weakly blocked by a magnesium ion or the magnesium block is nonexistent, depending on the region where the astrocyte is found, and the resting membrane potential of astrocytes is hyperpolarized in comparison to neurons. The theory that glutamate overstimulation of ionotropic receptors leads to excitotoxic neural cell death has been criticized, as AMPARs have been shown to quickly become desensitized to long glutamate exposure (Dzamba, Honsa, & Anderova, 2013). However, NMDARs show almost no desensitization to glutamate (Verkhratsky & Kirchhoff, 2007). This property, combined with their hyperpolarized glial resting membrane potential in comparison to neurons and lack of magnesium ion block, creates a vulnerability to significantly increased Ca²⁺ influx as a result of excessive extracellular glutamate concentration. This is significant because a positive correlation between the concentration of glutamate and the intensity of the Ca²⁺ influx in astrocytes has been observed by scientists. In vitro, astrocytes stimulated with glutamate have been shown to have increased cell death as well (Lee, Ting, Adams, Brew, Chung, & Guillemin, 2010).

In the presence of high levels of serum glutamate, NMDARs on astrocyte endfeet will be over activated, thus allowing dangerously high levels of Ca²⁺ to enter the cell. Such an influx of Ca²⁺ into an astrocyte is known to induce the vesicular release of glutamate from the astrocyte into the extracellular space (Malarkey & Parpura, 2008). Excessive extracellular glutamate in turn causes further damage to astrocytes by impairing astrocytic glutamate transporters and by inducing a fatal influx of calcium in both neurons and astrocytes. Studies have shown that excitatory amino acid transporter 2 (EAAT2) is responsible for about 90% of glutamate uptake in astroglial cells, making it a key glutamate transporter (Kim, Lee, Kegelman, Su, & Das, 2011). In response to an excess of glutamate in the extracellular space, EAAT2 on the astrocytic membrane works overtime to take up glutamate. However, with the persistent overload of glutamate, EAAT2 eventually becomes dysfunctional. Li, in 1997, found that of the four glutamate transporters, EAAT1 through EAAT4, EAAT2 was affected the most significantly in Alzheimer's disease (AD) patients. He noted an 85% loss of EAAT2 in AD patients (Li, Mallory, Alford, Tanaka, & Masliah, 1997). This loss of EAAT2 is disastrously detrimental to astrocytes and to surrounding neuronal and glial cells. Under normal physiological conditions, the role of astrocytes is to remove glutamate from the extracellular space, particularly synaptic clefts, and store the majority of the brain's glutamate. In fact, there is 10,000 times more glutamate in astrocytes than in the extracellular space (Ganel & Rothstein, 1999). When EAAT2 becomes dysfunctional, astrocytes can no longer take up glutamate or maintain glutamate homeostasis in the ECF. As a result, NMDARs on astrocytes that modulate glutamate levels around neuronal synapses and on post-synaptic neurons themselves become overstimulated. In 1994, Ulas et al. conducted an autoradiographic study on the binding of excitatory amino acid receptors in Parkinson's (PD) and Alzheimer disease patients. He found that in both PD and AD patients, there was a significant increase of binding to NMDARs (Ulas, Weihmuller, Brunner, Joyce, Marshall, & Cotman, 1994).

This increased binding of glutamate to NMDARs in both astrocytes and post-synaptic neurons can result in neuronal and glial cell death through the following mechanism, as explained by Dzamba, Honsa, and Anderova: the overactivation of NMDARs leads to an influx of Ca²⁺, which is taken up by mitochondria, which then become depolarized. This promotes the production of reactive oxygen species that can damage mitochondrial processes and the cell's ability to regulate its intracellular Ca²⁺, ultimately resulting in necrotic cell death. If the influx of Ca²⁺ through the NMDARs is less intense, apoptosis rather than necrosis results as the mitochondria becomes only partially depolarized. This allows for enough ATP to support the process of apoptosis (Dzamba, Honsa, & Anderova, 2013). Because astrocytes and neurons are physically close, the release of glutamate from astrocytes during Ca²⁺ influx affects both surrounding astrocytes and neurons, leading to neuronal excitotoxicity as the NMDARs in neurons become overstimulated by the extracellular glutamate and experience apoptotic or necrotic cell death just as astrocytes. Although usually dopamine protects neurons from glutamate-induced excitotoxicity by modulating Ca²⁺ signaling (Vaarmann, Kovac, Holmstrom, Gandhi, & Abramov, 2013), it has been found that an increase in NMDAR binding correlates with decreased binding at dopamine transporters and consequentially dopamine imbalances (Ulas, Weihmuller, Brunner, Joyce, Marshall, & Cotman, 1994). Therefore, calcium's homeostatic safety net, dopamine, is also inversely affected by the overstimulation of NMDAR. Both neurons and astrocytes are, therefore, directly and detrimentally impacted by persistent conditions of extracellular glutamate toxicity.

Unfortunately, the death of astrocytes and neurons due to extracellular glutamate toxicity creates a problem bigger than just their own death. The effects of excessive glutamate go beyond the direct chain of events of: serum glutamate overstimulating NMDARs in astrocytes endfeet, causing extreme Ca²⁺ influx which releases intracellular glutamate from the astrocyte and depolarizes mitochondria, leading to necrotic or apoptotic cell death. As mentioned earlier, a key role of astrocytic endfeet is to maintain the BBB through the secretion of growth factors that regulate the endothelial tight junctions. Unfortunately, when astrocytes die, the astrocytic endfeet are no longer able to maintain the BBB. Therefore, simply put, the death of astrocytes due to extracellular glutamate results in the loss of BBB integrity and an increase in BBB permeability.

There is a second pathway in which excess extracellular glutamate, which results from the vesicular release of glutamate from dysfunctional astrocytes, increases BBB permeability. Excess extracellular glutamate in the brain not only eliminates the protective role of astrocytes, but also directly impacts the tight junctions of endothelial cells. In a recent study, Vazana perfused the cortical area with glutamate and discovered through the use of a fluorescent tracer that BBB permeability increased (Vazana, et al., 2016). Through a series of tests, Vanza determined that excess extracellular glutamate over activates NMDA receptors on endothelial cells, which results in an influx of Ca²⁺, which then induces the production of nitric oxide (NO). NO then spreads to other endothelial cells through gap junctions and activates guanylyl cyclase to create cyclic guanosine monophosphate (cGMP). cGMP rearranges tight junction proteins, which ultimately makes the BBB more permeable. Therefore, we see that excessive glutamate in the extracellular space increases BBB permeability directly by manipulating tight cell junctions and indirectly by damaging astrocytes thereby preventing them from protecting the BBB.

There is also a third pathway in which extracellular glutamate increases BBB permeability. Ca²⁺ is an important factor both intracellularly and extracellularly to regulate tight junctions in the BBB and different molecules that modulate BBB permeability use intracellular Ca²⁺ to do so. An increase in extracellular Ca²⁺ correlates with decreased BBB permeability (Banerjee & Bhat, 2007). If Ca²⁺ influx is being abnormally increased by excessive extracellular glutamate binding to NMDARs, it follows that there is less extracellular Ca²⁺ available to help maintain and regulate BBB permeability and integrity. In fact, in normal physiology, the brain uses Ca²⁺ influx through NMDARs on astrocytes to make the BBB more permeable in order to increase brain oxygen levels (Mishra, Reynolds, Chen, Gourine, Rusakov, & Attwell, 2016). It seems as though in diseased conditions, the body uses this same mechanism to increase permeability of tight junctions. Therefore, when an increase in the permeability of tight junctions is combined with someone who has excessively high levels of serum glutamate, glutamate may inadvertently flow in through the BBB along with oxygen.

This consequential increase in permeability, which occurs through glutamate-induced: astrocytic death, endothelial tight junction rearrangement and/or decrease of extracellular Ca²⁺, opens the floodgates for substances that would normally be blocked from entering the brain, creating a positive feedback loop in which more serum glutamate passes the barrier, thus compounding the existing toxicity. The harmful effects of other substances that are now able to enter through the more permeable BBB also cannot be overlooked. The validity of our working model has been collaborated with studies by Leibowitz and associates; namely, when attempts are made to reduce the level of blood serum glutamate, regardless of the mechanism involved, a decreased blood glutamate concentration is associated with an improved neurological outcome (Leibowitz, Boyko, Shapira, & Zlotnik, 2012).

Thus, in light of this pathway, it is evident that elevated plasma glutamate is a significant factor in causing glutamate toxicity. However, the question remains: What causes elevated concentrations of glutamate in the blood? For humans, glutamate is obtained primarily from food. Glutamate is the most abundant amino acid in the human diet. It is consumed both in natural, as well as in many processed or hydrolyzed foods, and is used as an additive and flavor-enhancing ingredient in the form of monosodium glutamate (MSG).

In healthy subjects, much of the glutamate that is consumed in food is converted to glutamine within the gut, i.e. that portion of the gastrointestinal tract running from the pyloric sphincter of the stomach to the anus and, in humans, is comprised of the small and large intestines. The gut's microvilli then transfers glutamine and residual glutamate into the bloodstream. No toxic effects were observed in a study where 12.75 g of free glutamate was fed daily to young boys, showing that orally administered free glutamate is efficiently removed in healthy subjects (Nakagawa, Takahashi, & Suzuki, 1960). Other studies have also confirmed that plasma glutamate levels are not affected during diurnal meal intake. Similarly, when high protein meals are given to healthy human subjects, these meals failed to raise plasma glutamate, although there is an increase in glutamine, demonstrating that there is more efficient absorption of glutamine compared to glutamate across the gut mucosa (Palmer, Rossiter, Levin, & Oberholzer, 1973).

The gut is able to perform such a feat because in it dwells more than 100 trillion microorganisms, known collectively as the microbiome. These microorganisms, living in and on the human body, perform vital functions such as: synthesizing vitamins, aiding digestion, and developing and maintaining the immune system. The metabolism of glutamate to glutamine occurs primarily through glutamine synthetase-producing bacteria in the human small intestine. These bacteria are generally gram-positive bacteria including most species of Lactobacillus, such as L. plantarum, and gram-negative bacteria such as Escherichia coli, Bacteroides fragilis, Pseudomonas and Klebsiella. Glutamine Synthetase (GS) produced by these bacteria in the intestines is a vital enzyme for converting dietary glutamate into glutamine in the small intestine. The role of intestinal GS is incredibly significant in maintaining homeostatic levels of serum glutamate because its only purpose is to convert dietary glutamate into glutamine. No other enzyme in the intestine can perform such a function. Therefore, the presence and health of the gut's GS producing bacteria is paramount to maintaining homeostatic levels of glutamate in blood.

A deficiency or disruption of these resident bacteria due to gut dysbiosis leads to an impaired gut with digestive abnormalities. Dysbiosis is an imbalance in the gut flora caused by too few beneficial bacteria and an overgrowth of undesired bacteria, yeast, and/or parasites. Dysbiosis, therefore, results in a loss of GS activity and consequent insufficient and inefficient metabolism of dietary glutamate. This causes elevated blood free glutamate levels which are often many times greater than the serum basal levels of healthy subjects. Consequentially, intestinal homeostasis of the microbiome has been observed to play an essential role in neurological diseases such as amyotrophic lateral sclerosis (ALS) (Fang, 2015), Alzheimer's disease (Bhattacharjee & Lukiw, 2013), autism (Mulle, Sharp, & Cubells, 2013), schizophrenia (Nemani, Hosseini Ghomi, McCormick, & Fan, 2015), Parkinson's disease (Scheperjans, et al., 2014), multiple sclerosis (MS) (Westall, Molecular Mimicry Revisited: Gut Bacteria and Multiple Sclerosis, 2006), and schizophrenia (Nemani, Hosseini Ghomi, McCormick, & Fan, 2015). In fact, a study by Braniste and associates observed that germ free mice, which therefore are born with no microbiome, had increased BBB permeability. The increase in permeability was then improved and tight junction protein expression was upregulated when the mice were exposed to beneficial gut microbiota (Braniste, et al., 2014). An ALS transgenic SOD1-G93A mouse model displayed increased intestinal permeability and a shifted profile of the intestinal microbiome, suggesting a potentially unrecognized role of the microbiome in ALS (Shaoping Wu1, 2015). A similar study was reported for Parkinson's disease (Sampson, et al., 2016).

In our own investigation, we have seen similar results. Table 2 shows the comprehensive stool analysis report of an ALS patient. His report shows no growth of E. coli bacteria, one of the main glutamine synthetase bacteria in the small intestine, which in normal situations should be at 4+. Table 3 shows his fasting glutamate at 141 μmol/L, while normal fasting glutamate should be around 30 μmol/L. Normal plasma free glutamate of healthy people as per Peters study in 1969 should be 29.90 to 30.85 μmol/L (4.4-4.5 ppm) (Peters, Lin, Berridge, Cummings, & Chao, 1969). This example shows the ALS patient's serum glutamate to be 9 times higher than normal. The third column of this table shows his serum glutamate level at 271 μmol/L 90 minutes after feeding, while normal post-prandial serum glutamate should be around 60 μmol/L. This assumes the fasting serum glumate level to be less than or equal to 30 μmol/L, (fasting could be anywhere from 8 to 30 umol/L, and PPSG-Fasting Glutamate is expected to be lower than 30 umol/L, these are two separate marker) and that the difference between the post prandial serum glutamate level and the fasting serum glutamate level should not be greater than 30 μmol/L. Therefore, his post-prandial serum glutamate is about 4.5 times greater than normal levels. Table 3 shows another ALS patient's post-prandial serum glutamate at 340.4 μmol/L, more than 11 times higher than the serum glutamate level of healthy subjects. These elevated levels can lead to a cascading effect where the high concentration of free glutamate in the blood can breach the blood brain barrier leading to toxic conditions in the brain and the death of neurons. Therefore, it is our working model that gut dysbiosis can lead to the inability of the gut bacteria to produce GS and thus efficiently metabolize glutamate, which in turn causes an elevation of serum glutamate levels.

TABLE 2
Comprehensive Stool Analysis: Beneficial Flora for ALS patient # 1

| | Beneficial Bacterial Flora | | | | | |
|---|---|---|---|---|---|---|
| | Bacteroides | Bifidobacterium | Escherichia | Lactobacillus | Enterococcus | Clostridium |
| | fragilis | spp | Coli | spp. | spp. | Spp |
| Bacteriology | 3+ | 4+ | No | 4+ | 4+ | 2+ |
| Culture | | | Growth | | | |

Note:
Range is 1+ to 4+, where 4+ is normal and No growth being highly abnormal (arbitrary units)

TABLE 3
Fasting and Post Prandial Serum Glutamate
(Glu) Levels for 3 ALS Patients

| | | Normal | Post | Normal |
|---|---|---|---|---|
| | Fasting | Baseline | Prandial | Baseline |
| | umol/L | umol/L | umol/L | umol/L |
| Serum Glu Levels | 141 | 30 | 271 | 60 |
| Patient #1 | | | | |
| Serum Glu Levels | NA | NA | 340 | 60 |
| Patient # 2 | | | | |
| Serum Glu Levels | 33 | 30 | 184 | 60 |
| Patient # 3 | | | | |

NA: Data not available

The activity of GS in the gut, therefore, is paramount in preventing elevated glutamate serum levels and glutamate toxicity in the brain, and ultimately in preventing neurological disorders. Because GS activity in the intestine plays such an irreplaceable role in the homeostasis of the serum glutamate, it can potentially be used as a diagnostic tool for neurological disorders.

In 2001, Vermeiren et al. attempted to develop a biomarker for neurological diseases based on levels of GS in blood serum. He examined the GS levels in the blood serum of AD patients and control patients. However, he found that there was no statistically significant difference between the GS concentrations in AD and control subjects (Vermeiren, Le Bastard, Clark, Engelborghs, & De Deyn, 2011). He thereby ruled out GS in serum as an accurate biomarker. However, his search was slightly misguided in the fact that the majority of GS activity lies in the brain and in the microbiome of the gastrointestinal system. The level of GS in blood serum is not as telling as the levels of GS would be if measured in either the gut or the brain. In 1992, Gunnerson actually discovered that GS in the cerebral spinal fluid (CSF) could be used as a biomarker for neurological disease. He found that subjects with Alzheimer's disease (AD) had significantly more GS in their CSF than the control subjects. Of the 39 AD patients, 38 had GS in their CSF. Of the 44 controls, 1 had GS in their CSF (Gunnerson & Haley, 1992). Therefore, it can be seen that GS, if observed in the right location, can be used as an effective diagnostic tool for neurological disease. However, this form of testing is invasive, costly, dangerous, and lacks potential as a preventive diagnostic tool. Measuring levels of GS in the gut would also be both invasive and impractical given that GS in the gut is only produced by bacteria and these bacteria are only active in the presence of dietary glutamate. Furthermore, it would be costly and impractically complicated to quantify GS activity due to the large number of and complex interactions between the different species of microorganisms in the gut's microbiome.

### Glutamic Acid, Glutamate, Glutamine and Glutamine Synthetase

Glutamic acid is a naturally occurring alpha-amino acid having the chemical formula C₅H₉O₄N and corresponding to the following chemical structure for the L, i.e. the S, stereoisomer of glutamic acid.

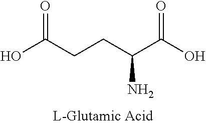

Glutamate is the main neurotransmitter of the human central nervous system and is the most abundant free amino acid in the system. Glutamate accounts for approximately 90 percent of the total neurotransmitter activity in the brain.

In its solid form and at slightly acidic pH values, glutamic acid exists as the zwitterion, corresponding to the following chemical structure.

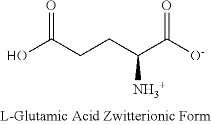

Glutamic acid is used by most living organisms in the biosynthesis of proteins. In humans it is considered a non-essential amino acid because it can be synthesized by the human body. Glutamic acid is widely found in a variety of proteins, including many food products such as meats, fish, dairy products, eggs, and soy protein. The sodium salt, monosodium glutamate, is used as a seasoning and flavor enhancer for foods.

The glutamate anion can be depicted by the following chemical structure

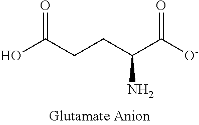

or by the overall, singly negative zwitterion

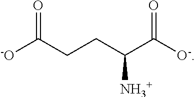

In the human body and most mammals, glutamic acid is metabolized to glutamine. The enzyme glutamine synthetase catalyzes the condensation of glutamate and ammonia to form glutamine as depicted by the following reaction.

Glutamate+ATP+NH₃→Glutamine+ADP+Phosphate

Glutamine synthetase enzyme (GS) is found in small quantities in the brain, kidney, liver, skeletal muscles and the heart. But the bulk of the enzymatic activity occurs in the small intestines of humans through the microbiome, which is capable of producing glutamine synthetase during digestion of proteins. However, for a variety of reasons, some subjects are not able to adequately metabolize dietary glutamate to glutamine, resulting in a deficiency of glutamine synthetase activity when compared to baseline levels of healthy subjects.

### Glutamine Synthetase-Producing Bacteria

The metabolism of dietary glutamate to glutamine in the human small intestines occurs primarily through glutamine synthetase-producing bacteria such as gram-positive bacteria including Butyrivibro fibrisolvens, many species of Lactobacillus such as Lactobacillus plantarum and gram-negative bacteria such as E. coil, Bacteriodes fragilis, Pseudomonas, and Klebsiella. Glutamine synthetase produced by these bacteria in the intestines is a vital enzyme for converting most of the glutamate from food sources into glutamine. A deficiency or disruption of these resident bacteria due to gut dysbiosis leads to an impaired gut with digestive abnormalities, notably abnormally elevated glutamate in the blood after a protein meal.

### Role of Glutamine Synthetase Bacteria in Central Nervous System Disorders

Many neurological patients complain about digestion and gut issues. With the displacement of resident glutamine synthetase bacteria, it is our hypothesis, corroborated by our clinical observations in this invention, that the capacity to metabolize glutamate in food could be severely impaired leading to inefficiency in conversion of dietary glutamate to glutamine, thereby being detectable as a percentage of glutamine synthetase deficiency when the fasting and postprandial levels of glutamate are measured. The consequent elevated glutamate in the blood may lead to the breaching of the blood brain barrier resulting in the manifestation of neurological conditions (Mayhan & Didion, 1996). It is difficult and impractical to measure glutamine synthetase activity in the intestines and fruitless to quantify the level of the enzyme in the blood serum. The current embodiment is designed as a simpler way to measure glutamine synthetase activity in a human subject as a biomarker for predicting the onset of or propensity for developing a central nervous system (CNS), psychotic, or related disorder, associated with glutamate toxicity. The method is also useful for designing regimens for modulating serum glutamate levels in a subject to treat or prevent such a disorder.

### Diagnostic Advantages of Glutamine Synthetase Deficiency Over Serum Glutamate

While it would be ideal to simply measure the levels of glutamine synthetase in a subject's gut, it is difficult to measure glutamine synthetase levels in the gut directly, as the complexity of the microbiome means a complete and accurate profile of a given subject's gut would be an expensive and laborious effort. Thus, it is infeasible as a diagnostic test.

Due to the impracticality of direct measurement, many studies instead look at serum glutamate levels in subjects. As referenced previously, elevated serum glutamate levels have been linked to neurological conditions in subjects of previous studies. However, we posit that the outlined model to calculate percentage of glutamine synthetase deficiency is superior to simply looking at serum glutamate levels. Quantifying glutamine synthetase deficiency more accurately evaluates and predicts severity and onset of neurological conditions and can be used as preventative early detection of neurological conditions.

For serum glutamate to reach high enough levels to be diagnostically abnormal, it can be inferred that the root cause of this elevated reading had to have been affecting the subject for a prolonged period of time. Therefore, while elevated serum glutamate can and has been linked to neurological conditions, it is not ideal as a diagnostic measure. If a subject shows elevated serum glutamate levels, the damage caused by this has likely been taking place for a while.

On the other hand, measuring the glutamine synthetase deficiency levels of a patient has the advantage of early detection capabilities. It is a deficiency of glutamine synthetase that eventually leads to elevated serum glutamate. Even before the problem progresses to the point where serum glutamate levels become elevated, the method outlined in this document can detect that danger. With this method, vulnerability to neurological conditions can be detected and disease progression can be predicted before a neurological disorder develops in the subject, posing a huge preventative benefit.

To perform the methods described herein, a blood sample can be obtained from a subject in need and the marker in the biological sample can be measured via methods known in the art, such as an immunoassay, e.g. ELISA (enzyme-linked immunosorbent assay). In some embodiments, two blood samples are obtained from a subject at two different time points e.g. a first fasting time point and after oral administration of an aqueous solution or suspension comprising glutamic acid (glutamate) a second postprandial time point. A subject in a fasting state is preferably fasted, except for water, for a period of at least about 12 hours. A second postprandial time point is about 15 minutes to about 90 minutes after the oral administration of an aqueous solution or suspension comprising glutamic acid (glutamate).

As known in the art, glutamic acid (glutamate) can be present in a variety of protein-rich food source. Therefore, in some embodiments, the aqueous solution or suspension as used herein can be a nutritional composition comprising a diary protein source such as whey protein, casein protein, or soy protein. Commercially available examples of such nutritional composition include for example Osmolite (Abbott).

In certain embodiments, the aqueous solution or suspension comprises the equivalent of about 70 mg/kg to about 225 mg/kg based on the weight of the subject of glutamic acid (glutamate). In one example, the aqueous solution or suspension comprises the equivalent of about 150 mg/kg based on the weight of the subject of glutamic acid (glutamate).

In certain embodiments, the aqueous solution or suspension comprises the equivalent of about 10 grams of glutamic acid (glutamate).

In certain embodiments, the aqueous solution or suspension comprises a digestible protein. For example, the aqueous solution or suspension is a solution or suspension of whey protein. Preferably, the aqueous solution or suspension is substantially free of glutamine.

In certain embodiments, the aqueous suspension or solution comprises about 75 [preferably about 50] grams of the whey protein suspended or dissolved in about 200 to about 250 ml of water or fruit juice. Whey protein is preferred, because it contains glutamate and not glutamine, as do other forms of protein.

Specifically, the subject during the collection of both samples, the first (fasting) blood sample and the second (post prandial) blood sample, is not allowed to urinate, because doing so will lower the serum glutamate right away and artificially distort (lower the level through excretion), resulting in voided tests. The subject is only allowed to urinate right before the collection of the first (fasting) blood sample and right after the collection of the second (post prandial) blood sample. Moreover, catharized subjects should be excluded or specifically controlled for.

Blood sample can be obtained by different ways known in the art e.g. peripheral vein puncture (venipuncture). The blood samples can be subjected to processing with an anti-coagulate, centrifugation and/or deproteinization, to obtain protein free serum samples. The serum samples as obtained can be analyzed for the glutamate level in each sample by methods known in the art such as an immunoassay, e.g. ELISA.

In certain embodiments, if the difference between the serum glutamate level in the second sample to the serum glutamate level in the first sample is greater than a predetermined value, e.g. 30 μmol/liter of serum glutamate,—the subject is deemed as having intestinal glutamine synthetase activity deficiency or an abnormal elevated (excess) serum glutamate or having or at risk for a disease associated therewith or its progression.

In certain embodiments, if the percent intestinal glutamine synthetase deficiency is greater than a predetermined value e.g. 19.11 percent, the subject is deemed as having intestinal glutamine synthetase activity deficiency or an abnormal elevated (excess) serum glutamate or having or at risk for a disease associated therewith or its progression.

After a subject has been determined as having intestinal glutamine synthetase activity deficiency or an abnormal elevated (excess) serum glutamate or having or at risk for a disease associated therewith or its progression, the subject can be subjected to a further test (such as a conventional physical examination, including imaging tests, e.g., X-ray mammograms, magnetic resonance imaging (MRI) or ultrasound to conform the disease occurrence and/or determine the stage/phase of progression.

In some embodiments, the methods described herein can further comprise treating the subject to at least enhance intestinal glutamine synthetase activity or lower an abnormal elevated (excess) serum glutamate or alleviate a symptom associated with the disease.

The present invention also provides a composition as a pharmaceutical composition for treatment.

In particular embodiments, a glutamine synthetase or an agent capable of increasing an intestinal glutamine synthetase activity can be used as an active ingredient to manufacture a medicament for treating intestinal glutamine synthetase activity deficiency or a disease associated therewith or preventing progression of such disease in a subject in need. Such agent can be a probiotic, optional with a prebiotic to adjust the population of non-pathogenic glutamine synthetase producing bacteria in the small intestines of the subject.

As used herein, “pharmaceutically acceptable” means that the carrier is compatible with the active ingredient in the composition, and preferably can stabilize said active ingredient and is safe to the individual receiving the treatment. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents. The composition of the present invention can provide the effect of rapid, continued, or delayed release of the active ingredient after administration to the patient.

According to the present invention, the form of said composition may be tablets, pills, powder, lozenges, packets, troches, elixers, suspensions, lotions, solutions, syrups, soft and hard gelatin capsules, suppositories, sterilized injection fluid, and packaged powder.

The composition of the present invention may be delivered via any physiologically acceptable route, such as oral, parenteral other than oral, fecal microbiotic transplants and suppository methods. Regarding parenteral administration, it is preferably used in the form of a sterile water solution, which may comprise other substances, such as salts or glucose sufficient to make the solution isotonic to blood. The water solution may be appropriately buffered (preferably with a pH value of 3 to 9) as needed. Preparation of an appropriate parenteral composition under sterile conditions may be accomplished with standard pharmacological techniques well known to persons skilled in the art, and no extra creative labor is required.

Also described herein is a kit for performing the method of the invention, which comprises an agent that is capable of specifically detecting glutamate in the samples. Such agent can be, for example, an antibody, to perform an immunoassay. An antibody as used herein can refer to an immunoglobulin molecule having the ability to specifically bind to a particular target antigen. An antibody as used herein includes not only intact (i.e. full-length) antibody molecules but also antigen-binding fragments thereof retaining antigen binding ability e.g. Fab, Fab′, F(ab′)2 and Fv. An antibody as used herein can include humanized antibodies, chimeric antibodies, diabodies, linear antibodies, single chain antibodies, or multispecific antibodies (e.g., bispecific antibodies). Antibodies as described herein are commercially available or can be made by methods known in the art e.g. by a hybridoma method.

In some embodiments, the immunoassay can be in a sandwich format. Particularly, the kit comprises a capture antibody paired with a detection antibody that comprises a detectable label such as an enzymatic label, a fluorescent label, a metal label and a radio label. In certain examples, the kit is an ELISA sandwich kit, comprising a microtiter plate with wells to which a capture antibody has been immobilized, a solution containing a detection antibody and a color developing reagent. Particularly, the kit may further comprise additional reagents or buffers, a medical device for collecting a biological sample form a subject, and/or a container for holding and/or storing the sample.

The detection assays can be carried out in other forms, for example, by using any hardware, a biochip, micro and nano-array technologies or equivalent, optionally in combination with chemical or radio isotope labeling technologies for automatic (automated) measurement of serum glutamate levels, and complete with hardware and software for measurements with computational output showing quantification, diagnostic range of intestinal glutamine synthetase deficiency levels.

In some examples, the kit may comprise a detection device configured to detect the results of the assay and produce a signal proportional to the glutamate level in each well; and a reader configured to read the signal and preferably further to indicate a positive result, when the difference between the serum glutamate level in the second sample to the serum glutamate level in the first sample is greater than a predetermined value, or the percent intestinal glutamine synthetase deficiency is greater than a predetermined value. The reader can be further configured to indicate intestinal glutamine synthetase activity deficiency or an abnormal elevated (excess) serum glutamate or having or at risk for a disease associated therewith or its progression. In some embodiments, the reader can indicate a negative result, when the difference between the serum glutamate level in the second sample to the serum glutamate level in the first sample is less than a predetermined value, or the percent intestinal glutamine synthetase deficiency is less than a predetermined value; and the reader can be further configured to indicate having no intestinal glutamine synthetase activity deficiency or having a normal level of serum glutamate or less likelihood of occurrence or risk for a disease associated with an abnormal elevated serum glutamate or its progression.

The kit can further comprise instructions for using the kit to detect glutamate levels in the samples and calculate to obtain the difference between the serum glutamate level in the second sample to the serum glutamate level in the first sample, or the percent intestinal glutamine synthetase deficiency.

### EXAMPLES

The following examples further describe and demonstrate embodiments within the scope of the present invention. The examples are given solely for purpose of illustration and are not to be construed as limitations of the present invention, as many variations thereof are possible without departing from the spirit and scope of the invention.

### Example 1

### Method for Monitoring Serum Glutamate Levels—Healthy Subject

The present method was applied to a subject (age 19, female) having no diagnosed neurological disorders and having a fasting serum glutamate concentration of 19.8 μmol/L. The postprandial serum glutamate level measured 60 minutes after taking 11.3 grams of dietary glutamate was 47.8 μmol/L, which was 28 μmol/L higher than fasting serum glutamate.

The present method demonstrates that the difference of postprandial to fasting glutamate levels is within the normal range of 30 μmol/L. The 1969 study by Peters describes normal levels for healthy individuals (Peters, Lin, Berridge, Cummings, & Chao, 1969). With the formula, the subject's percent deficiency is brought to 0%, showing that the subject is metabolizing dietary glutamate to glutamine normally and has no evidence of glutamine synthetase deficiency.

### Example 2

### Method for Monitoring Serum Glutamate Levels—Subject with a Minor Lifestyle Related Glutamine Synthetase Deficiency

The present method was applied to a subject (age 23, male) having no diagnosed neurological disorders and having a fasting serum glutamate concentration of 23.8 μmol/L. The postprandial serum glutamate level measured 60 minutes after taking 11.3 grams of dietary glutamate was 54.6 μmol/L, which was 30.8 μmol/L higher than fasting serum glutamate.

The present method demonstrates that the difference of postprandial to fasting glutamate levels is slightly outside of the normal range of 30 μmol/L. With this value, the subject's percent deficiency cannot be brought to 0% and the values are inputted into the formula. The difference between the measurements of glutamate is divided by the difference between the measurements of glutamate of the subject with the highest recorded value for this difference, which is 187 μmol/L, with 30 μmol/L subtracted from both values. The resulting percentage comes out to be a mere 0.51% glutamine synthetase deficiency. This shows that the subject is borderline healthy since a small value as 0.51% correlates to their body's slight inability to metabolize consumed glutamate at a healthy rate. This can be interpreted as not a glutamine synthetase related deficiency but a lifestyle related deficiency where the subject's dietary pattern explains the slight reading, or could be within the range of standard error.

### Example 3

### Method for Monitoring Serum Glutamate Levels—Mild Glutamine Synthetase Deficiency

The present method was applied to a subject (age 19, female) having no diagnosed neurological disorders and having a fasting serum glutamate concentration of 20.2 μmol/L. The postprandial serum glutamate level measured 60 minutes after taking 11.3 grams of dietary glutamate was 75 μmol/L, which was 54.8 μmol/L higher than fasting serum glutamate.

The present method demonstrates that the difference of postprandial to fasting glutamate levels is outside the normal range of 30 μmol/L. With this value, the subject's percent deficiency cannot be brought to 0% and the values are inputted into the formula. The difference between the measurements of glutamate is divided by the difference between the measurements of glutamate of the patient with the highest recorded value for this difference, which is 187 μmol/L, with 30 μmol/L subtracted from both values. The resulting percentage comes out to be a 15.8% glutamine synthetase deficiency. Although the fasting glutamate is a healthy level below 30 μmol/L, the subsequent increase in glutamate indicates a mild glutamine synthetase deficiency.

### Example 4

### Method for Monitoring Serum Glutamate Levels—Moderate Glutamine Synthetase Deficiency

The present method was applied to a subject (age 21, male) having no diagnosed neurological disorders and having a fasting serum glutamate concentration of 88.0 μmol/L. The postprandial serum glutamate level measured 60 minutes after taking 11.3 grams of dietary glutamate was 161.3 μmol/L, which was 73.3 μmol/L higher than fasting serum glutamate.

The present method demonstrates that the difference of postprandial to fasting glutamate levels is outside the normal range of 30 μmol/L. With this value, the subject's percent deficiency cannot be brought to 0% and the values are inputted into the formula. The difference between the measurements of glutamate is divided by the difference between the measurements of glutamate of the patient with the highest recorded value for this difference, which is 187 μmol/L, with 30 μmol/L subtracted from both values. The resulting percentage is a 27.58% glutamine synthetase deficiency. The subject has a fasting glutamate level almost 3 times the healthy norm. This is indicative of the subject consuming more glutamate than their body can metabolize and excrete. This analysis is supported by the subject's high protein diet and low daily water intake. The difference between the postprandial and fasting glutamate levels for the subject was more than double the expected 30 μmol/L increase. This is indicative that aside from the high levels of glutamate in the body, the subject also suffers from a deficiency of glutamine synthetase. If the subject fails to either change their diet or replenish their body's glutamine synthetase, then they would be on track to eventually compromise the integrity of their blood brain barrier, thus becoming at risk for developing symptoms of neurological disorders.

### Example 5

### Method for Monitoring Serum Glutamate Levels—High Glutamine Synthetase Deficiency Due to Alkaline Water

The present method was applied to a subject (age 21, female) having no diagnosed neurological disorders and having a fasting serum glutamate concentration of 53.5 μmol/L. The postprandial serum glutamate level measured 60 minutes after taking 11.3 grams of dietary glutamate was 163.4 μmol/L, which was 109.9 μmol/L higher than fasting serum glutamate.

The present method demonstrates that the difference of postprandial to fasting glutamate levels is more than three times the normal range of 30 μmol/L. With this value, the subject's percent deficiency cannot be brought to 0% and the values are inputted into the formula. The difference between the measurements of glutamate is divided by the difference between the measurements of glutamate of the patient with the highest recorded value for this difference, which is 187 μmol/L, with 30 μmol/L subtracted from both values. The resulting percentage comes out to be a 50.89% glutamine synthetase deficiency. This shows that the subject is on track to be at risk in the future since a high value as 50.89% correlates to their body's inability to metabolize consumed glutamate at a healthy rate and is in the ballpark of ALS patients who are older.

Upon further investigation, the subject revealed that she has been drinking alkaline water almost exclusively for the past 3 years. It was later confirmed that the water from her sources have been measured to be at a pH of 8.6. Another subject (age 22, male) without diagnosed neurological disorders reported a similarly high percent deficiency of 50.1% and later also revealed that he had started drinking alkaline water exclusively since 2 months prior. The Lactobacillus strain responsible for producing glutamine synthetase is known to thrive in a pH level of 6.5 and the basicity of alkaline water has been recorded to increase the pH to levels around 8.6. There is record of a subject (age 57, male), who intentionally drank alkaline water with a pH range between 8.6 to 9.0 exclusively while also taking two teaspoons of L. plantarum once every week. Within 4 months, he developed extreme insomnia, early peripheral neuropathy, mild discoordination in both feet and panic attacks without a triggering cause. When receiving his CDSA, it was found that there was no growth recorded for Lactobacillus. Even three consecutive antibiotic treatments will only bring down the growth of Lactobacillus from a healthy 4+ to 1+ or 2+. This patient admitted to having never taken antibiotics in the last 50 years.

### Summary of Results:

Table 4 provides a summary of the results.

TABLE 4

| | | | Diet | | | | |
|---|---|---|---|---|---|---|---|
| GluPP − | | | Related | | | | GluPP − |
| GluF | | GSD | Glutamate | | | | GluF |
| (umol/L) | % of GSD | Problem | toxicity | Interpretation | Remark | Application | (umol/L) |
| <=30 | 0% | No | No | Perfectly | Normal | Use this | <=30 |
| | | | | Healthy | metabolism | condition to | |
| | | | | | or excretion | rule out | |
| | | | | | according to | both GS | |
| | | | | | the 1969 | deficiency & | |
| | | | | | Peters study | diet related | |
| | | | | | | glutamate | |
| | | | | | | toxicity | |
| <=30 | 0% | No | Mild | Borderline | The body is | Use this | <=30 |
| | | | | Healthy | unable to | condition to | |
| | | | | | excrete | monitor | |
| | | | | | serum | early case | |
| | | | | | glutamate | of diet | |
| | | | | | due to | related | |
| | | | | | chronic | glutamate | |
| | | | | | dehydration | toxicity | |
| <=30 | (Glupp − | No | Moderate | Early stage | Patient is | Use this | <=30 |
| | GluF − 30)/ | | | Glutamate | consuming | condition to | |
| | Max(Glupp − | | | toxicity | more | identify diet | |
| | GluF − | | | | glutamate | related | |
| | 30) | | | | than their | glutamate | |
| | | | | | kidney can | toxicity | |
| | | | | | excrete it | | |
| <=30 | (Glupp − | No | Severe | late stage | Chronic | Use this to | <=30 |
| | GluF − 30)/ | | | Glutamate | dehydration | rule out non | |
| | Max(Glupp − | | | toxicity | together with | GSD | |
| | GluF − | | | | high protein | related | |
| | 30) | | | | diet could be | severe | |
| | | | | | responsible | glutamate | |
| | | | | | for this | toxicity | |
| | | | | | condition. | | |
| >60 | (Glupp − | No | Acute | Acute | Severe | Use this to | >60 |
| | GluF − 30)/ | | | Glutamate | dehydration | rule out non | |
| | Max(Glupp − | | | toxicity | together with | GSD | |
| | GluF − | | | | extreme | related | |
| | 30) | | | | protein diet, | acute | |
| | | | | | even without | glutamate | |
| | | | | | GSD | toxicity | |
| | | | | | problem, the | | |
| | | | | | gut bacteria | | |
| | | | | | are | | |
| | | | | | overwhelmed | | |
| | | | | | by too much | | |
| | | | | | dietary | | |
| | | | | | glutamate | | |
| | | | | | and could | | |
| | | | | | not | | |
| | | | | | metabolize it | | |
| | | | | | fast enough | | |
| | | | | | during the 2- | | |
| | | | | | 3-hour | | |
| | | | | | digestion | | |
| | | | | | process. | | |
| 31-60 | (Glupp − | Mild | No | Borderline | Mild GSD | Use this | 31-60 |
| | GluF − 30)/ | | | Healthy | problem | condition to | |
| | Max(Glupp − | | | | | monitor | |
| | GluF − | | | | | patient with | |
| | 30) | | | | | mild GSD | |
| | | | | | | problems. | |
| 61-90 | (Glupp − | Moderate | No | Early stage | Moderate | Use this | 61-90 |
| | GluF − 30)/ | | | Glutamate | GSD | condition to | |
| | Max(Glupp − | | | toxicity | problem | identify | |
| | GluF − | | | | | patient with | |
| | 30) | | | | | moderate | |
| | | | | | | GSD | |
| | | | | | | related | |
| | | | | | | glutamate | |
| | | | | | | toxicity. | |
| 90-150 | (Glupp − | Severe | No | late stage | Severe GSD | This group | 90-150 |
| | GluF − 30)/ | | | Glutamate | problem | of patients | |
| | Max(Glupp − | | | toxicity | | should | |
| | GluF − | | | | | respond to | |
| | 30) | | | | | a probiotic | |
| | | | | | | treatment | |
| >150 | (Glupp − | Acute | No | Acute | Acute GDS | This group | >150 |
| | GluF − 30)/ | | | Glutamate | problem, the | of patients | |
| | Max(Glupp − | | | toxicity | body is | will need to | |
| | GluF − | | | | unable to | limit their | |
| | 30) | | | | excrete | glutamate | |
| | | | | | serum | intake for a | |
| | | | | | glutamate | probiotic | |
| | | | | | even when | treatment to | |
| | | | | | well hydrated | work | |
| | | | | | | effectively | |

### Example 6

### Assessment of Glutamine Synthetase Deficiency in Subjects with Various Neurological Disorders

The methods of the present invention were used to determine the percent glutamine synthetase deficiency in a group of 37 subjects, both male and female, ranging in age from 31 to 95 years old with neurological disorders. Fasting serum glutamate (Glut) and post prandial serum glutamate (Glupp) levels were measured for each subject. The difference in levels, i.e. Glupp−Gluf was then calculated for each subject. The percent glutamine synthetase deficiency (% GSD) was then determined from this difference as follows:

A value of 30 umol/L, which is considered to be a normal value of serum glutamate, was subtracted from the difference in Glupp−Gluf that was calculated for each subject. If the resulting value was zero or less for that subject, the % GSD is assigned a value of zero %. If the resulting value was greater than zero, this result was then divided by 157 uMol/L, which is highest increase from fasting to post prandial serum glutamate among all the data sets collected (recognizing that a higher value could be observed from a larger data set), and which is considered to be a pathologically elevated and undesirable level of glutamate. Multiplying this quotient by 100 therefore provides the % GSD for the subject. Data are presented in Table 5.

TABLE 5
Patients with Neurological Disorders

| | Age | | Gluf | Glupp | Glupp- | |
|---|---|---|---|---|---|---|
| Subject | years | Health Status | μMol/L | μMol/L | Gluf | % GSD |
| 1 | 69 | ALS | 55.4 | 143.2 | 87.80 | 36.82% |
| 2 | 66 | ALS limb | 41 | 181 | 140 | 70.06% |
| 3 | 77 | ALS bulbar | 33 | 184 | 151 | 77.07% |
| 4 | 56 | ALS limb | 74 | 245 | 171 | 89.81% |
| 5 | 66 | ALS bulbar | 33 | 73 | 40 | 6.37% |
| 6 | 62 | ALS Limb | 103 | 137 | 34 | 2.55% |
| 7 | 70 | ALS limb | 50 | 89 | 39 | 5.73% |
| | | LMN | | | | |
| 8 | 70 | ALS limb | 48 | 127 | 79 | 31.21% |
| 9 | 39 | ALS limb | 76 | 263 | 187 | 100.00% |
| 10 | 50 | FALS bulbar | 63 | 205 | 142 | 71.34% |
| 11 | 58 | early onset PD | 29 | 142 | 113 | 52.87% |
| 12 | 52 | ALS limb | 32 | 121 | 89 | 37.58% |
| 13 | 62 | ALS bulbar | 32 | 118 | 86 | 35.67% |
| 14 | 56 | ALS limb | 40 | 124 | 84 | 34.39% |
| 15 | 68 | ALS limb | 39 | 117 | 78 | 30.57% |
| 16 | 68 | ALS limb | 41 | 110 | 69 | 24.84% |
| 17 | 73 | ALS pulm | 55 | 109 | 54 | 15.29% |
| 18 | 76 | bulbar ALS | 41 | 92 | 51 | 13.38% |
| | | not in study | | | | |
| 19 | 83 | ALS limb | 34 | 76 | 42 | 7.64% |
| 20 | 69 | Bulbar FALS | 68 | 109 | 41 | 7.01% |
| 21 | 53 | early case | 86.2 | 174.2 | 88 | 36.94% |
| | | of AD | | | | |
| 22 | 95 | Alzheimer | 37.9 | 106.8 | 68.9 | 24.78% |
| 23 | 64 | Pseudobulbar | 49.8 | 70.5 | 20.7 | 0% |
| | | Palsy | | | | |
| 24 | 49 | Probably ALS | 93.4 | 94.3 | 0.9 | 0% |
| 25 | 57 | ALS limb | 36 | 66 | 30 | 0% |
| 26 | 47 | ALS limb | 54 | 66 | 12 | 0% |
| 27 | 67 | ALS limb | 114 | 109 | −5 | 0% |
| 28 | 37 | ALS limb | 29 | 49 | 20 | 0% |
| 29 | 31 | vet with fascics | 55 | 43 | −12 | 0% |
| 30 | 58 | ALS limb | 82 | 53 | −29 | 0% |
| 31 | 74 | ALS bulbar | 108 | 70 | −38 | 0% |
| 32 | 82 | ALS | 22 | 48 | 26 | 0% |
| 33 | 77 | ALS limb | 24 | 46 | 22 | 0% |
| 34 | 52 | Parkinson | 42 | 63 | 21 | 0% |
| | | Disease | | | | |
| 35 | 47 | ALS limb | 29 | 43 | 14 | 0% |
| 36 | 62 | FTLD/ALS | 83 | 91 | 8 | 0% |
| 37 | 59 | ALS limb/pulm | 64 | 96 | 32 | 1.27% |
| Average | 62 | — | 53.96 | 109.57 | 55.60 | 21.98% |

Summary of data from Table 5.

| ALS Patients | | | | |
|---|---|---|---|---|
| with | | | | |
| Neurological | | ALS with | ALS without | % |
| Disorders | Overall | GluTox | GluTox | Increase |
| Sample Size | 37 | 22 | 15 | — |
| Average | 53.96 | 50.52 | 59.01 | −14.39% |
| fasting | | | | |
| Glutamate | | | | |
| Average PPSG | 109.57 | 138.46 | 67.19 | 106.09% |
| Average PPSG − | 55.60 | 87.94 | 8.17 | 975.95% |
| Fasting | | | | |
| Glutamate | | | | |
| Average | 21.98% | 36.91% | 0.08% | 36.82% |
| % GSD among | | | | |
| all ALS patient | | | | |
| Average Age | 62 | 65 | 58 | — |

### Example 7

### Assessment of Glutamine Synthetase in Generally Healthy Subjects

The methods of the present invention (see Example 5) were used to determine the percent glutamine synthetase deficiency in a group of 26 generally healthy subjects (also including some indicated as overweight), both male and female, ranging in age from 20 to 32 years old. Data are presented in Table 6.

TABLE 6

| | | | Gluf | Glupp | Glupp- | |
|---|---|---|---|---|---|---|
| Subject | Age years | Health Status | μMol/L | μMol/L | Gluf | % GSD |
| 1 | 23 | Healthy | 44.6 | 176.8 | 132.2 | 65.10% |
| 2 | 22 | Healthy | 83.1 | 202.1 | 119 | 56.69% |
| 3 | 22 | Healthy | 53.5 | 163.4 | 109.9 | 50.89% |
| 4 | 23 | Healthy | 38.3 | 148.1 | 109.8 | 50.83% |
| 5 | 23 | Healthy | 38.7 | 119.5 | 80.8 | 32.36% |
| 6 | 22 | Healthy | 88.0 | 161.3 | 73.3 | 27.58% |
| 7 | 21 | Severely | 45.9 | 111.5 | 65.6 | 22.68% |
| | | Overweight | | | | |
| 8 | 23 | Healthy | 61.9 | 125.5 | 63.6 | 21.40% |
| 9 | 20 | Healthy | 20.2 | 75 | 54.8 | 15.80% |
| 10 | 27 | Slightly | 39.1 | 90 | 50.9 | 13.31% |
| | | Overweight | | | | |
| 11 | 21 | Healthy | 16.3 | 66.9 | 50.6 | 13.12% |
| 12 | 24 | Healthy | 48.6 | 89.5 | 40.9 | 6.94% |
| 13 | 24 | Healthy | 23.8 | 54.6 | 30.8 | 0.51% |
| 14 | 20 | Healthy | 17.1 | 45.7 | 28.6 | 0% |
| 15 | 21 | Healthy | 19.8 | 47.8 | 28 | 0% |
| 16 | 25 | Healthy | 41.6 | 62.7 | 21.1 | 0% |
| 17 | 31 | Healthy | 20.2 | 41.3 | 21.1 | 0% |
| 18 | 25 | Healthy | 15.9 | 36.6 | 20.7 | 0% |
| 19 | 22 | Healthy | 23.5 | 40.4 | 16.9 | 0% |
| 20 | 32 | Healthy | 17.0 | 31.9 | 14.9 | 0% |
| 21 | 26 | Healthy | 26.4 | 40.6 | 14.2 | 0% |
| 22 | 23 | Healthy | 19.6 | 29.1 | 9.5 | 0% |
| 23 | 25 | Healthy | 24.3 | 32.1 | 7.8 | 0% |
| 24 | 21 | Healthy | 17.1 | 22.7 | 5.6 | 0% |
| 25 | 31 | Healthy | 15.9 | 24.4 | 8.5 | 0% |

Summary of data from Table 6.

| Healthy and | | Healthy with | Healthy without | % |
|---|---|---|---|---|
| Young People | Overall | GluTox | GluTox | Increase |
| Sample Size | 25 | 12 | 13 | — |
| Average | 34.42 | 48.18 | 21.71 | 121.96% |
| fasting | | | | |
| Glutamate | | | | |
| Average PPSG | 81.58 | 127.47 | 39.22 | 224.98% |
| Average PPSG − | 47.16 | 79.28 | 17.52 | 352.65% |
| Fasting | | | | |
| Glutamate | | | | |
| Average | 15.09% | 31.39% | 0.04% | 31.35% |
| % GSD among | | | | |
| all ALS patient | | | | |
| Average Age | 24 | 23 | 25 | — |

Summary of data from Table 6.

| | |
|---|---|
| Sample Size of Healthy and Young people less than 30 year | 25 |
| old | |
| Average fasting Glutamate | 34.42 |
| Average PPSG | 81.58 |
| Average PPSG − Fasting Glutamate | 47.16 |
| Average % GSD | 15.09% |
| Average Age | 24 |
| Sample size among healthy & young people with | 12 |
| GluTox Problem | |
| Average fasting Glutamate among those with GluTox problem | 48.18 |
| Average PPSG among those with GluTox problem | 127.47 |
| Average PPSG − Fasting Glutamate among those with | 79.28 |
| GluTox problem | |
| Average % GSD among those with GluTox problem | 31.39% |
| Average Age | 23 |
| Sample size among healthy & young people without | 13 |
| GluTox Problem | |
| Average fasting Glutamate among those without | 21.71 |
| GluTox problem | |
| Average PPSG among those without GluTox problem | 39.22 |
| Average PPSG − Fasting Glutamate among those | 17.52 |
| without GluTox problem | |
| Average % GSD among those without GluTox problem | 0.04% |
| Average Age | 25 |

### REFERENCES

Andreaou, E., Kapaki, E., Kokotis, P., Paraskevas, G. P., Katsaros, N., Libitaki, G., et al. (2008). Plasma Glutamate and Glycine Levels in Patients with Amyotrophic Lateral Sclerosis. In Vivo, 22(137-142), 137-41.
Banerjee, S., & Bhat, M. A. (2007). Neuron-Glial Interactions in Blood-Brain Barrier. Annual Review of Neuroscience, 235-258.
Bhattacharjee, S., & Lukiw, W. J. (2013). Alzheimer's disease and the microbiome. Frontiers in Cellular Neuroscience, 7(Article 153).
Braniste, V., Al-Asmakh, M., Kowal, C., Anuar, F., Abbaspour, A., Toth, M., et al. (2014). The gut microbiota influences blood-brain barrier permeability in mice. Science Translational Medicine.
Cabezas, R., Avila, M., Gonzalez, J., Baez, E., Garcia-Segura, L. M., El-Bacha, R. S., et al. (2014 [symbol] 4-August). Astrocytic modulation of blood brain barrier: perspectives on Parkinson's disease. Frontiers in Cellular Neuroscience, 1-11.
Campos, F., Sobrino, T., Ramos-Cabrer, P., Argibay, B., Agulla, J., Perez-Mato, M., et al. (2011). Neuroprotection by glutamate oxaloacetate transaminase in ischemic stroke: an experimental study. Journal of Cerebral Blood Flow & Metabolism, 31(1378-1386).
Dzamba, D., Honsa, P., & Anderova, M. (2013). NMDA Receptors in Glial Cells: Pending Questions. Current Neuropharmacologu, 250-262.
Fang, X. (2015). Potential role of gut microbiota and tissue barriers in Parkinson's disease and amyotrophic lateral sclerosis. International Journal of Neuroscience.
Ganel, R., & Rothstein, J. (1999). Glutamate Transporter Dysfunction and Neuronal Death. In Handbook of Experimental Pharmacology (Vol. 141, pp. 471-487). Springer, Berlin, Heidelberg.
Gunnerson, D., & Haley, B. (1992). Detection of glutamine synthetase in the cerebral spinal fluid of Alzheimer diseased patients: A potential diagnostic biochemical marker. Proc. Natl. Acad. Sci. USA, 11949-11953.
Ivanovaa, S. A., Boykoa, A. S., Yu., F., Krotenkoa, N., Semkea, A., & Bokhana, N. A. (2014). Glutamate concentration in the serum of patients with schizophrenia. Procedia Chemistry, 10(80-85), 80-85.
Iwasaki, Y., Ikeda, K., Shojima, T., & Kinoshita, M. (1992). Increased plasma concentrations of aspartate, glutamate and glycine in Parkinson's disease. Neuroscience Letters, 145(175 177), 175-7.
Kim, K., Lee, S.-G., Kegelman, T. P., Su, Z.-Z., & Das, S. K. (2011). Role of Excitatory Amino Acid Transporter-2 (EAAT2) and Glutamate in Neurodegeneration: Opportunities for Developing Novel Therapeutics. J Cell Physiology.
Lee, M.-C., Ting, K. K., Adams, S., Brew, B. J., Chung, R., & Guillemin, G. J. (2010). Characterization of the Expression of NMDA Receptors in Human Astrocytes. Plos One, 1-11.
Leibowitz, A., Boyko, M., Shapira, Y., & Zlotnik, A. (2012). Blood Glutamate Scavenging: Insight into Neuroprotection. International Journal of Medical Sciences, 13(10041-10066), 10041-10066.
Li, S., Mallory, M., Alford, M., Tanaka, S., & Masliah, E. (1997). Glutamate transporter alterations in Alzheimer disease are possibly associated with abnormal APP expression. Journal of Neuropathology and Experimental Neurology, 901-911.
Malarkey, E. B., & Parpura, V. (2008). Mechanisms of glutamate release from astrocytes. Neurochem International, 142-154.
Mayhan, W. G., & Didion, S. P. (1996). Glutamate-Induced Disruption of the Blood-Brain Barrier in Rats. Stroke, 27(965-970), 959-9.
Mishra, A., Reynolds, J., Chen, Y., Gourine, A., Rusakov, D., & Attwell, D. (2016). Astrocytes mediate nerovascular signaling to capillary pericytes but not to arterioles. Nature Neuroscience, 19, 1619-1627.
Miulli, D. E., Norwell, D. Y., & Schwartz, F. N. (1993). Plasma concentrations of glutamate and its metabolites in patients with Alzheimer's disease. The Journal of the American Osteopathic Association, 93(6), 670-6.
Mulle, J. G., Sharp, W. G., & Cubells, J. F. (2013). The Gut Microbiome: A New Frontier in Autism Research. Current Psychiatry Reports, 15(Article 337).
Nakagawa, I., Takahashi, T., & Suzuki, T. (1960). Amino Acid Requirements of Children. J. Nutrition, 70, 176-181.
Nemani, K., Hosseini Ghomi, R., McCormick, B., & Fan, X. (2015). Schizophrenia and the gut-brain axis. Progress in Neuro-Psychopharmacology and Biological Psychiatry, 56(155-160), 155-160.
Palmer, T., Rossiter, M., Levin, B., & Oberholzer, V. G. (1973). The Effect of Protein Loads on Plasma Amino Acid Levels. Clinical Science and Molecular Medicine, 45(827-832), 827-832.
Peters, J. H., Lin, S. C., Berridge, B. J., Cummings, J. G., & Chao, W. R. (1969). Amino Acids, Including Asparagine and Glutamine, in Plasma and Urine of Normal Human Subjects. Experimental Biology and Medicine, 131.
Plaitakis, A., & Caroscio, J. T. (1987). Abnormal Glutamate Metabolism in Amyotrophic Lateral Sclerosis. Annals of Neurology, 22(575-579), 575-9.
Rainesalo, S., Keranen, T., Palmio, J., Peltola, J., Oja, S. S., & Saransaari, P. (2004). Plasma and Cerebrospinal Fluid Amino Acids in Epileptic Patients. Neurochemical Research, 29(No 1, 319-324), 319-324.
Sampson, T. R., Debelius, J. W., Thron, T., Janssen, S., Shastri, G. G., Ilhan, Z. E., et al. (2016). Gut Microbiota Regulate Motor Deficits and Neuroinflammation in a Model of Parkinson's Disease. Cell (1469-1480), 1469-1480.
Scheperjans, F., Aho, V., Pereira, P. A., Koskinen, K., Paulin, L., Pekkonen, E., et al. (2014). Gut Microbiota Are Related to Parkinson's Disease and Clinical Phenotype. Movement Disorders, 00(00).
Shaoping Wu1, J. Y.-g. (2015). Leaky intestine and impaired microbiome in an amyotrophic lateral sclerosis mouse model. Physiological Reports, 10.
Shimmura, C., Shiro, S., Tsuchiya, K. J., Hashimoto, K., Ohno, K., Matsuzaki, H., et al. (2011). Alteration of Plasma Glutamate and Glutamine Levels in Children with High-Functioning Autism. PloS One, 6(10).
Stegink, L. D. et al., Factors Affecting Plasma Glutamate Levels in Normal Adults Subjects, pages 333-351, page 345, in Glutamic Acid: Advances in Biochemistry and Physiology, edited by L. J. Filer, Jr., et al. Raven Press, NY 1979).
Ulas, J., Weihmuller, F. B., Brunner, L. C., Joyce, J. N., Marshall, J. F., & Cotman, C. W. (1994). Selective Increase of NMDA-Sensitive Glutamate binding in the Striatum of Parkinson's Disease, Alzheimer's Disease and Mixed Parkinson's Disease/Alzheimer's Disease Patients: An Autoradiographic Study. The Journal of Neuroscience, 6317-6324.
Vaarmann, A., Kovac, S., Holmstrom, K. M., Gandhi, S., & Abramov, A. Y. (2013). Dopamine protects neurons against glutamate-induced excitotoxicity. Cell Death and Disease, 1-6.
Vazana, U., Veksler, R., Pell, G., Prager, O., Fassler, M., Chassidim, Y., et al. (2016 [symbol] 20-July). Glutamate-Mediated Blood-Brain Barrier Opening: Implications for Neuroprotection and Drug Delivery. Journal of Neuroscience, 36(29), 7727-7739.
Verkhratsky, A., & Kirchhoff, F. (2007). NMDA Receptors in Glia. The Neuroscientist, 28-37.
Vermeiren, Y., Le Bastard, N., Clark, C. M., Engelborghs, S., & De Deyn, P. P. (2011). Serum Glutamine Synthetase Has No Value as a Biomarker for Alzheimer's Disease. Neurochem Research, 1858-1862.
Westall, F. C. (2006). Molecular Mimicry Revisited: Gut Bacteria and Multiple Sclerosis. Journal of Clinical Microbiology, 44(6), 2099-104.
Westall, F. C., Hawkins, A., Ellison, G. W., & Myers, L. W. (1980). Abnormal glutamic acid metabolism in multiple sclerosis. Journal of the Neurological Sciences, 47(353-364), 353-364.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

Equivalents

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps or components, it is also contemplated that the methods and systems consist essentially of, or consist of, the recited steps or components. Furthermore, the order of steps or order for performing certain actions is immaterial as long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as composed of the components prior to mixing, because upon mixing certain components can further react or be transformed into additional materials.

All percentages and ratios used herein, unless otherwise indicated, are by weight.

1. A method for treating a human patient for intestinal glutamine synthetase activity deficiency, said deficiency indicating the onset of or propensity for developing a central nervous system (CNS), psychotic, or neurological disorder, comprising the steps of:
(I) determining the intestinal glutamine synthetase activity in the human patient at two or more selected time points, comprising the steps of:
(a) fasting the patient, except for water, for a period of at least about 12 hours;
(b) withdrawing by venipuncture from the patient a first (fasting) blood sample;
(c) transferring the first blood sample to a first container, optionally containing an anticoagulant pre-cooled between about 0° C. to about 5° C.;
(d) orally administering to the patient an aqueous solution or suspension comprising the equivalent of about 5 to about 15 grams of glutamic acid (glutamate);
(e) about 15 minutes to about 90 minutes after the administration of the aqueous solution or suspension of step (d), withdrawing by venipuncture from the patient a second (post prandial) blood sample;
(f) transferring the second blood sample to a second container, optionally containing an anticoagulant pre-cooled between about 0° C. to about 5° C.;
(g) centrifuging each of the first and second blood samples to separate the blood serum from the blood platelets in the blood samples, to provide a first (fasting) serum sample and a second (post prandial) serum sample,
(h) deproteinization of each of the first serum sample and the second serum sample by the addition of a deproteinizing agent to each of the serum samples;
(i) centrifuging each of the serum samples from step (h) to separate the protein from the serum in the samples, to provide a first (fasting) protein free serum sample and a second (post prandial) protein free serum sample;
(j) analyzing the first and second protein free serum samples to determine the serum glutamate level of each sample; and
(k) comparing the serum glutamate levels from step (j) to indirectly determine the intestinal glutamine synthetase activity of the patient; and
(II) treating the human patient for intestinal glutamine synthetase activity deficiency if the difference between the serum glutamate level in the second sample to the serum glutamate level in the first sample is greater than a predetermined value.

2-27. (canceled)

28. A method according to claim 1 wherein the predetermined value is 30 μmol/liter of serum glutamate.

29-84. (canceled)

85. A method according to claim 1, wherein the patient is other than a catharized patient, wherein the patient is not allowed to urinate from the time the first (fasting) blood sample is withdrawn until the second (post prandial) blood sample is withdrawn, and wherein in step (k) the intestinal glutamine synthetase activity of the patient is determined from the difference between the serum glutamate levels of each sample.

86. A method according to claim 1, wherein in step (k) the intestinal glutamine synthetase activity of the patient is determined from the ratio of the serum glutamate levels of each sample.

87. A method according to claim 1, wherein in step (k) the intestinal glutamine synthetase activity for the patient is determined as a ratio of intestinal glutamine synthetase deficiency by (A) determining the difference between the serum glutamate level in the second sample and the serum glutamate level in the first sample, (B) subtracting 30 μmol/liter from the result of step (A), and (C) dividing the result of step (B) by the approximate maximum serum glutamate level for a sample population.

88. A method according to claim 88, comprising the further step (D) of step (k) of multiplying the result of step (C) of step (k) by 100 to obtain a percentage of intestinal glutamine synthetase deficiency.

89. A method according to claim 1, wherein in step (d) the aqueous solution or suspension comprises the equivalent of about 70 mg/kg to about 225 mg/kg based on the weight of the patient of glutamic acid (glutamate).

90. A method according to claim 1 wherein in step (d) the aqueous suspension or solution is a solution or suspension of whey protein.

91. A method according to claim 91 wherein in step (d) the aqueous suspension or solution of the whey protein is substantially free of glutamine.

92. A method according to claim 92 wherein in step (d) the aqueous suspension or solution comprises about 75 grams of the whey protein suspended or dissolved in water or fruit juice.

93. A method according to claim 1 wherein the time in step (e) is about 60 minutes.

94. A method according to claim 1 wherein in step (II) the method of treating excess serum glutamate is by increasing the intestinal glutamine synthetase activity in the patient.

95. A method according to claim 95 wherein the method in step (I) comprises orally administering a probiotic to adjust the population of non-pathogenic glutamine synthetase producing bacteria in the small intestines of the patient.

96. A method according to claim 96 wherein said bacteria is a Lactobacillus species.

97. A method according to claim 97 wherein said Lactobacillus species is Lactobacillus plantarum.

98. A method according to claim 97 wherein the method in step (II) comprises orally administering a probiotic with a prebiotic to adjust the population of non-pathogenic glutamine synthetase producing bacteria in the small intestines of the patient.

99. A method for treating a central nervous system or psychotic disorder comprising the method of claim 1.

100. A method according to claim 100 wherein the neurological or psychotic disorder is selected from Alzheimer's disease, amyotrophic lateral sclerosis, autism, cerebral atrophy, dementia, epilepsy, major depressive disorders, multiple sclerosis, obsessive compulsive disorder, Parkinson's disease, peripheral neuropathy, restless legs syndrome, schizophrenia, stiff man syndrome, and stroke.

101. A method for treating a human patient for intestinal glutamine synthetase activity deficiency, said deficiency-indicating the onset of or propensity for developing a central nervous system (CNS), psychotic, or neurological disorder, comprising the steps of:
I. determining the intestinal glutamine synthetase activity in the human patient at two or more selected time points, comprising the steps of:
(a) fasting the patient, except for water, for a period of at least about 12 hours;
(b) withdrawing by venipuncture from the patient a first (fasting) blood sample;
(c) transferring the first blood sample to a first container, optionally containing an anticoagulant pre-cooled between about 0° C. to about 5° C.;
(d) orally administering to the patient an aqueous solution or suspension comprising the equivalent of about 5 to about 15 grams of glutamic acid (glutamate);
(e) about 15 minutes to about 90 minutes after the administration of the aqueous solution or suspension of step (d), withdrawing by venipuncture from the patient a second (post prandial) blood sample;
(f) transferring the second blood sample to a second container, optionally containing an anticoagulant pre-cooled between about 0° C. to about 5° C.;
(g) centrifuging each of the first and second blood samples to separate the blood serum from the blood platelets in the blood samples, to provide a first (fasting) serum sample and a second (post prandial) serum sample,
(h) deproteinization of each of the first serum sample and the second serum sample by the addition of a deproteinizing agent to each of the serum samples;
(i) centrifuging each of the serum samples from step (h) to separate the protein from the serum in the samples, to provide a first (fasting) protein free serum sample and a second (post prandial) protein free serum sample;
(j) analyzing the first and second protein free serum samples to determine the serum glutamate level of each sample; and
(k) comparing the serum glutamate levels from step (j) to indirectly determine the intestinal glutamine synthetase activity of the patient; and
(II) treating the human patient for intestinal glutamine synthetase activity deficiency if the percent intestinal glutamine synthetase deficiency is greater than a predetermined value.

102. A method according to claim 101 wherein the predetermined value is 19.11 percent.

103. A method according to claim 101, wherein the patient is other than a catharized patient, wherein the patient is not allowed to urinate from the time the first (fasting) blood sample is withdrawn until the second (post prandial) blood sample is withdrawn, and wherein in step (k) the intestinal glutamine synthetase activity of the patient is determined from the difference between the serum glutamate levels of each sample.

104. A method according to claim 101, wherein in step (k) the intestinal glutamine synthetase activity of the patient is determined from the ratio of the serum glutamate levels of each sample.

105. A method according to claim 101, wherein in step (k) the intestinal glutamine synthetase activity for the patient is determined as a ratio of intestinal glutamine synthetase deficiency by (A) determining the difference between the serum glutamate level in the second sample and the serum glutamate level in the first sample, (B) subtracting 30 μmol/liter from the result of step (A), and (C) dividing the result of step (B) by the approximate maximum serum glutamate level for a sample population.

106. A method according to claim 106, comprising the further step (D) of step (k) of multiplying the result of step (C) of step (k) by 100 to obtain a percentage of intestinal glutamine synthetase deficiency.

107. A method according to claim 101, wherein in step (d) the aqueous solution or suspension comprises the equivalent of about 70 mg/kg to about 225 mg/kg based on the weight of the patient of glutamic acid (glutamate).

108. A method according to claim 101 wherein in step (d) the aqueous suspension or solution is a solution or suspension of whey protein.

109. A method according to claim 108 wherein in step (d) the aqueous suspension or solution of the whey protein is substantially free of glutamine.

110. A method according to claim 110 wherein in step (d) the aqueous suspension or solution comprises about 75 grams of the whey protein suspended or dissolved in water or fruit juice.

111. A method according to claim 101 wherein the time in step (e) is about 60 minutes.

112. A method according to claim 101 wherein in step (II) the method of treating excess serum glutamate is by increasing the intestinal glutamine synthetase activity in the patient.

113. A method according to claim 113 wherein the method in step (II) comprises orally administering a probiotic to adjust the population of non-pathogenic glutamine synthetase producing bacteria in the small intestines of the patient.

114. A method according to claim 114 wherein said bacteria is a Lactobacillus species.

115. A method according to claim 115 wherein said Lactobacillus species is Lactobacillus plantarum.

116. A method according to claim 115 wherein the method in step (II) comprises orally administering a probiotic with a prebiotic to adjust the population of non-pathogenic glutamine synthetase producing bacteria in the small intestines of the patient.

117. A method for treating a central nervous system or psychotic disorder comprising the method of claim 101.

118. A method according to claim 117 wherein the neurological or psychotic disorder is selected from Alzheimer's disease, amyotrophic lateral sclerosis, autism, cerebral atrophy, dementia, epilepsy, major depressive disorders, multiple sclerosis, obsessive compulsive disorder, Parkinson's disease, peripheral neuropathy, restless legs syndrome, schizophrenia, stiff man syndrome, and stroke.

119. A method for treating a human patient for intestinal glutamine synthetase activity deficiency, said deficiency-indicating the onset of or propensity for developing a central nervous system (CNS), psychotic, or neurological disorder, comprising the steps of:
I. determining the intestinal glutamine synthetase activity in the human patient at two or more selected time points, comprising the steps of:
(i) providing a first (fasting) blood sample which is obtained from the subject at a first time point in a fasting state, wherein the subject is preferably fasted, except for water, for a period of at least about 12 hours;
(ii) providing a second (post prandial) blood sample which is obtained from the subject at a second time point that is about 15 minutes to about 90 minutes after oral administration of an aqueous solution or suspension comprising the equivalent of about 5 to about 15 grams of glutamic acid (glutamate) to the subject in the fasting state of step (i);
(iii) transferring the first blood sample to a first container, optionally containing an anticoagulant pre-cooled between about 0° C. to about 5° C.;
(iv) transferring the second blood sample to a second container, optionally containing an anticoagulant pre-cooled between about 0° C. to about 5° C.;
(v) centrifuging each of the first and second blood samples to separate the blood serum from the blood platelets in the blood samples, to provide a first (fasting) serum sample and a second (post prandial) serum sample,
(vi) deproteinization of each of the first serum sample and the second serum sample by the addition of a deproteinizing agent to each of the serum samples;
(vii) centrifuging each of the serum samples from step (vi) to separate the protein from the serum in the samples, to provide a first (fasting) protein free serum sample and a second (post prandial) protein free serum sample;
(viii) analyzing the first and second protein free serum samples to determine the serum glutamate level of each sample; and
(ix) comparing the serum glutamate levels from step (viii) to indirectly determine the intestinal glutamine synthetase activity of the patient; and
(II) treating the human patient for intestinal glutamine synthetase activity deficiency if the difference between the serum glutamate level in the second sample to the serum glutamate level in the first sample is greater than a predetermined value or if the percent intestinal glutamine synthetase deficiency is greater than a predetermined value.

120. A method according to claim 119 wherein the predetermined value is 30 μmol/liter of serum glutamate for the difference between the serum glutamate level in the second sample compared to the first value.

121. A method according to claim 119 wherein the predetermined value is 19.11 percent for the percent intestinal glutamine deficiency.
